(19) United States
(12) Patent Application Publication
(10) Pub. No.: US 2025/0226109 A1
(43) Pub. Date: Jul. 10, 2025

(54) ARTIFICIAL INTELLIGENCE-BASED SYSTEM AND METHOD FOR AUTOMATED PREVENTATIVE HEALTH SCREENING AND DATA-DRIVEN HEALTH RISK ANALYSIS

(71) Applicants: FEROZ SYED, Carmel, IN (US); FNU MOHAMMED KHADER ALISHA BIN AHMED, Carmel, IN (US)

(72) Inventors: FEROZ SYED, Carmel, IN (US); FNU MOHAMMED KHADER ALISHA BIN AHMED, Carmel, IN (US)

(21) Appl. No.: 19/013,043

(22) Filed: Jan. 8, 2025

Related U.S. Application Data
(60) Provisional application No. 63/618,968, filed on Jan. 9, 2024.

(51) Int. Cl. G16H 50/30; G06F 21/62; G16H 10/20

(52) U.S. Cl. CPC G16H 50/30; G06F 21/6254; G16H 10/20

(57) ABSTRACT

Exemplary embodiments of the present disclosure are directed towards artificial intelligence-based system for automated preventative health screening and data-driven health risk analysis. The system integrates Generative AI with the CDC's Syndemic Model to enable anonymous, stigma-free health screening and care linkage. The system leverages oral health as neutral entry point to assess interconnected risks across sexual, mental, and behavioral health domains. Using Large Language Models trained on validated sources and AI-based image analysis of oral and skin abnormalities, system computes “Chance screening Scores” to classify risks for communicable (e.g., HIV, STIs, Mpox) and non-communicable diseases (e.g., diabetes, hypertension). Accessible through mobile apps/QR codes without requiring login, system generates Unique IDs for test kits, telehealth services, and rewards. Features include actionable recommendations, gamified Stigma Meter, geographic insights, language translation, and data control options. This innovative solution fosters informed decision-making, reduces stigma, and empowers users to manage their health confidentially and effectively.

### CROSS-REFERENCE TO RELATED APPLICATIONS

This patent application claims priority benefit of U.S. Provisional Patent Application No. 63/618,968, entitled “ARTIFICIAL INTELLIGENCE ENABLED SYSTEM FOR USER HEALTHCARE ASSESSMENT AND METHOD EMPLOYED THEREOF”, filed on 9 Jan. 2024. The entire contents of the patent application are hereby incorporated by reference herein in its entirety.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever.

### TECHNICAL FIELD

The disclosed subject matter relates generally to the field of healthcare informatics and artificial intelligence. More particularly, the present disclosure relates to an artificial intelligence-based system and method for automated preventative health screening and data-driven health risk analysis. Additionally, the present disclosure focuses on the early identification of oral manifestations associated with HIV/STD, mental health conditions and streamlines the process of directing exposed individuals to suitable testing, care and management interventions as needed.

### BACKGROUND

The persistent challenge of Human Immunodeficiency Virus (HIV) remains a significant public health concern in the United States, with approximately 1.2 million Americans living with HIV, and alarmingly, about 14% are unaware of their infection. The Centers for Disease Control and Prevention (CDC) recommends regular HIV screening for individuals aged 13 to 64, with higher-risk groups advised to undergo annual screening. However, suboptimal screening rates, especially within high-risk populations, contribute to delayed diagnoses and pose substantial hurdles in controlling the spread of the virus.

Additionally, individuals dealing with communicable diseases, especially HIV/STDs, encounter multiple challenges when initiating their journey toward care, particularly in oral and mental health. Recent reports from the International AIDS Society (IAS) and the World Health Organization (WHO) in 2023 highlight a concerning threefold increase in the risk of Human Papillomavirus (HPV) related oropharyngeal cancers among those with HIV. Furthermore, the landscape is marked by a noteworthy 7% surge in cases of Sexually Transmitted Diseases (STDs), compounding the health risks faced by this vulnerable population. Adding to the complexity, a shortage of adequately trained healthcare professionals poses a significant impediment to the provision of comprehensive care. Additionally, the financial barrier, denoted by the pivotal role of costs in accessing timely screening and diagnosis, further accentuates the multifaceted challenges individuals with HIV grapple with as they navigate their healthcare journey. Addressing these issues requires innovative solutions that integrate advanced technologies and methodologies to ensure accessible, timely, and comprehensive healthcare for this population.

The intersection of technology and healthcare is at a critical juncture, as advancements in artificial intelligence (AI) present unprecedented opportunities to address long-standing challenges in healthcare delivery. Among these challenges, stigma remains one of the most significant barriers to accessing care in domains such as HIV prevention, sexual health, and mental health. Fear of judgment, privacy concerns, and fragmented care models deter individuals from seeking timely interventions, exacerbating health disparities, particularly in vulnerable populations.

Compounding this issue, research reveals that during and after the COVID-19 pandemic, over 50% of Americans deferred their dental care, disproportionately impacting individuals living with HIV. This population faces distinctive oral health challenges due to the virus's impact on the immune system and the potential side effects of medications. The postponement of dental care exacerbates their vulnerability to oral health issues, emphasizing the need for innovative solutions to bridge healthcare gaps. Oral Health: Oral manifestations, such as candidiasis or gum disease, often serve as early indicators of systemic conditions like HIV and diabetes (NIH, 2022). Sexual Health: Stigma around sexually transmitted infections (STIs) not only delay diagnosis but also creates a cascade of mental health challenges, including anxiety and depression (Lancet Public Health, 2021). Mental Health: Poor mental health exacerbates risk behaviors and reduces adherence to preventive measures, such as condom use and medication adherence (APA, 2022).

Interestingly, despite the overall delay in healthcare visits during the pandemic, accessing dental services was perceived as more convenient and efficient compared to visiting primary care doctors. Factors such as the perceived urgency of dental issues, ease of scheduling, and the perceived safety of dental settings during the pandemic may have contributed to this trend. Stigma is a significant impediment to healthcare access, particularly in HIV and sexual health services. Nearly 40% of U.S. adults avoid HIV testing due to fear of judgment or breaches of privacy (CDC, 2023). Young adults and marginalized communities are disproportionately affected, resulting in undiagnosed and untreated cases.

Recognizing the intricate connection between oral health and overall well-being, the collaboration between primary healthcare providers and oral health specialists has become increasingly vital. Oral health conditions can serve as indicators of underlying systemic issues, including communicable infections such as HIV/STDs. Prompt identification and education about oral conditions related to these infections are paramount. Strengthening the partnership between primary care physicians and dental specialists can facilitate early detection, timely intervention, and education on oral health issues associated with systemic infections like HIV/STDs.

The disconnection between oral, sexual, and mental health services contributes to suboptimal outcomes. Only 25% of traditional healthcare systems address the interplay between these domains (NIH, 2022). This fragmentation leads to missed opportunities for early intervention and comprehensive care. Oral health is a critical, yet often overlooked, indicator of overall health. Oral conditions such as candidiasis and lesions may signal immunosuppression, a hallmark of HIV. Poor oral health is linked to cardiovascular disease, diabetes, and other systemic conditions. Research indicates that untreated mental health conditions deter individuals from seeking sexual health services, perpetuating adverse health outcomes (Lancet Public Health, 2021). The stigma surrounding sexual health exacerbates mental health challenges such as anxiety and depression.

In the light of the aforementioned discussion, there exists a need for a system with novel methodologies that would overcome the above-mentioned challenges.

### SUMMARY

The following presents a simplified summary of the disclosure in order to provide a basic understanding of the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards an artificial intelligence-based system and method for automated preventative health screening and data-driven health risk analysis.

An objective of the present disclosure is directed towards a system that provides a holistic approach to healthcare, addressing oral, mental, and overall health through an integrated platform.

Another objective of the present disclosure is directed towards a system that facilitates anonymous and confidential interaction.

Another objective of the present disclosure is directed towards a system that utilizes images and user responses to assess risk, providing a risk score and treatment recommendations.

Another objective of the present disclosure is directed towards a system that seamlessly integrates with dental case management systems and web-based referral links.

Another objective of the present disclosure is directed towards a system that assists oral health providers and primary care providers (PCPs) in remote areas. Facilitates early identification of oral manifestations associated with HIV/STD.

Another objective of the present disclosure is directed towards a system that provides an intelligent virtual assistance (IVA) chatbot that provides risk scoring while maintaining user anonymity and confidentiality, fostering a safe environment for health-related queries.

Another objective of the present disclosure is directed towards a system that ensures consistent and interconnected data reporting, fulfilling the aim of empowering healthcare professionals with a unified view for more effective case management.

Another objective of the present disclosure is directed towards a system that promotes overall well-being by advocating for the improvement of public health, emphasizing the interconnection between oral, behavioral, mental, and sexual health.

Another objective of the present disclosure is directed towards a system that creates a medium for early detection and easy education tools, encouraging individuals to re-engage in care and fostering discrete surveillance techniques.

Another objective of the present disclosure is directed towards a system that simplifies the process of surveillance and care retention by providing coherent and interconnected systems that work in harmony, thus fulfilling the objective of reducing administrative complexities.

Another objective of the present disclosure is directed towards a system that specifically targets high-risk groups, such as young people, teenagers, and individuals living with HIV/STIs, providing accessible point-of-care tools for anonymous and confidential use.

Another objective of the present disclosure is directed towards a system that employs an innovative three-pronged strategy, including education (prevention), diagnosis (care), and contact tracing and linkage (surveillance) for managing infectious and communicable diseases.

Another objective of the present disclosure is directed towards a system that increases foot traffic for checkups in communities, especially among those who may not be aware of the connections between oral, sexual, and mental health.

Another objective of the present disclosure is directed towards a system that empowers healthcare providers, including case managers and surveillance staff, with a single system of care, streamlining workflows and improving overall efficiency.

Another objective of the present disclosure is directed towards a system that addresses financial barriers to healthcare by providing cost-effective access to timely screening and diagnosis, contributing to increased accessibility.

Another objective of the present disclosure is directed towards a system that proposes to bridge the gap between stigma and awareness, fostering connections to essential care and contributing to the reduction of stigma associated with certain health conditions.

Another objective of the present disclosure is directed towards a system that enhances public health understanding by promoting the interconnection between oral, behavioral, mental, and sexual health, fostering a more comprehensive and inclusive approach to healthcare.

Another objective of the present disclosure is directed towards a system that enhances accessibility for vulnerable populations, ensuring that individuals, including those living with HIV/STIs, have increased access to essential healthcare services.

Another objective of the present disclosure is directed towards a system that integrates smart reporting and telehealth services, providing a comprehensive approach to healthcare management, including anti-retro viral resistance (AVR) tracking.

Another objective of the present disclosure is directed towards advocating for timely screening and diagnosis, emphasizing the importance of early detection and intervention for infectious and communicable diseases.

Another objective of the present disclosure is directed towards promoting discrete surveillance techniques, encouraging individuals to actively participate in healthcare monitoring while maintaining privacy.

Another objective of the present disclosure is directed towards fostering user engagement in care, encouraging individuals to take proactive steps toward improving their overall health through the utilization of user-friendly tools and resources.

Another objective of the present disclosure is directed towards incorporating behavioral/mental health queries into the intelligent virtual assistance (IVA) chatbot, providing a more comprehensive evaluation of the user's health status.

Another objective of the present disclosure is directed towards a system that empowers case managers and surveillance staff, facilitating more efficient decision-making, and improving overall healthcare coordination.

Another objective of the present disclosure is directed towards an innovative approach for contact tracing, leveraging advanced technologies to enhance the efficiency and accuracy of contact identification and linkage.

Another objective of the present disclosure is directed towards providing tailored educational content through the chatbot, catering to the specific needs and queries of users related to oral, behavioral, mental, and sexual health.

Another objective of the present disclosure is directed towards a system that allows users to easily access the preventative health screening module (chatbot) by scanning a QR code or by downloading the preventative health screening module from Android/iOS stores, without requiring login or sign-up procedures.

Another objective of the present disclosure is directed towards offering syndemic gamified health screenings and insights by evaluating the interconnectedness of various health aspects, including oral health, behavioral health, sexual life, substance abuse, and their links to both communicable (e.g., COVID-19, STIs) and non-communicable diseases (e.g., Diabetes, Hypertension).

Another objective of the present disclosure is directed towards a system that allows users the ability to delete their personal information and close the system at any time, ensuring privacy and security, thus maintaining complete anonymity.

Another objective of the present disclosure is directed towards a system that offers extensive support and practical options, including peer support, claim and eligibility checks, preventive alerts, prescription reminders, chat replays, consultations, gifting test kits, sharing the app, guided testing, peer education, and rewards such as coupons for engaging in health-promoting activities, all within a gamified experience.

Another objective of the present disclosure is directed towards creating personalized care plans for healthcare providers such as case managers and providers, helping them make informed decisions and improving care quality for users.

Another objective of the present disclosure is directed towards a system that provides a holistic view of user health data for case managers (e.g., educators, infectious disease specialists), healthcare providers (e.g., doctors, dentists, nurses), and others, enabling them to manage comprehensive screening results effectively.

Another objective of the present disclosure is directed towards a system that requires case managers, providers, and healthcare staff to verify their profiles through their organization before accessing the preventative health screening module, ensuring that only authorized professionals can use the system for case management and healthcare decisions.

Another objective of the present disclosure is directed towards a system that includes real-time language translation and allows users to download smart conversational reports, making it accessible to a diverse user base.

Another objective of the present disclosure is directed towards a system that allows users to fill out eligibility forms in their preferred language, which are automatically submitted to case managers for approval through the preventative health screening module.

Another objective of the present disclosure is directed towards a system that includes a chat feature in the preventative health screening module where users can request dashboards, which the preventative health screening module generates as downloadable reports.

Another objective of the present disclosure is directed towards a system that leverages oral health as a stigma-free entry point to promote HIV/STI screening and related health risk awareness among young adults.

Another objective of the present disclosure is directed towards a system that facilitates stigma reduction through conversational AI, enabling young adults to approach healthcare discussions with reassurance and confidence.

Another objective of the present disclosure is directed towards a system that promotes early intervention and care by using innovative conversational tools to address HIV/STI-related stigma.

Another objective of the present disclosure is directed towards a system that employs generative AI to dismantle barriers to healthcare access, focusing on outreach, screening, and management solutions.

Another objective of the present disclosure is directed towards a system that prioritizes user privacy and accessibility, eliminating the need for users to navigate complex systems or share personal data.

Another objective of the present disclosure is directed towards a system that uses oral health as a neutral and stigma-free gateway to encourage engagement with sensitive health topics, including STI/HIV testing, PrEP (pre-exposure prophylaxis) uptake, and mental health support.

Another objective of the present disclosure is directed towards a system that normalizes healthcare engagement by framing discussions around oral health, fostering comfort and trust in addressing broader health concerns.

Another objective of the present disclosure is directed towards a system rooted in a syndemic framework, recognizing the interconnectedness of oral, sexual, and mental health within social and environmental contexts.

Another objective of the present disclosure is directed towards a system that delivers personalized health insights and behavioral nudges in real-time, empowering users to make informed health decisions while maintaining their anonymity.

Another objective of the present disclosure is directed towards a system that operates on a data-free model, eliminating fears of data misuse and fostering trust among users, especially in stigmatized health domains.

Another objective of the present disclosure is directed towards a system that not only empowers individuals to take control of their health but also drives systemic change by fostering community-level awareness and normalization of health-seeking behaviors.

Another objective of the present disclosure is directed towards leveraging AI-enabled chatbots to provide anonymous sexual and reproductive health (SRH) education, addressing design and implementation barriers while fostering user and community engagement.

Another objective of the present disclosure is directed towards a system that incorporates gamification strategies, such as the Stigma Meter, to engage users in reducing stigma by rewarding actionable behaviors, including completing screenings, sharing the platform, and accessing care resources.

Another objective of the present disclosure is directed towards a system that utilizes oral health as a stigma-free gateway for addressing sensitive topics like HIV and mental health through neutral discussions.

Another objective of the present disclosure is directed towards a system that enables anonymous and judgment-free conversations, fostering privacy and trust for users.

Another objective of the present disclosure is directed towards providing behavioral nudges, such as PrEP/PEP reminders, to guide users toward proactive health actions and preventive care.

Another objective of the present disclosure is directed towards ensuring complete anonymity by eliminating the need for sign-ups, data retention, and offering options to delete conversations post-use, thereby safeguarding user privacy.

Another objective of the present disclosure is directed towards connecting users with essential care resources, including at-home HIV test kits, counseling services, and safe sex supplies.

Another objective of the present disclosure is directed towards leveraging AI-driven image analysis to evaluate user-uploaded images of oral lesions and skin conditions, providing insights into gum health or potential skin abnormalities.

Another objective of the present disclosure is directed towards a system that educates users on risks such as HPV and oral cancer through personalized health risk analysis and care guidance.

Another objective of the present disclosure is directed towards a gamified engagement system that rewards users for completing health actions with discounts and perks, fostering sustained engagement.

Another objective of the present disclosure is directed towards a system that addresses the interconnected impacts of HIV, oral health, and mental health through an integrated, stigma-free platform.

Another objective of the present disclosure is directed towards a system that leverages a syndemic framework to align with public health goals by reducing stigma associated with HIV testing and mental health support.

Another objective of the present disclosure is directed towards a system that promotes early detection of systemic health risks through oral health screenings, facilitating proactive healthcare interventions.

Another objective of the present disclosure is directed towards a system that encourages holistic care pathways, bridging the gaps in traditional siloed healthcare systems for comprehensive health management.

Another objective of the present disclosure is directed towards a system that integrates multiple health domains into a single, user-friendly platform, enabling individuals to manage their health in a discreet, informed, and comprehensive manner.

Another objective of the present disclosure is directed towards a system with a mobile-first design and gamified engagement, specifically tailored to improve healthcare access for rural and underserved communities.

Another objective of the present disclosure is directed towards a system that provides anonymous, stigma-free access to testing resources and counseling services, addressing barriers to healthcare engagement.

Another objective of the present disclosure is directed towards a system that incentivizes participation through rewards and discounts, encouraging user engagement in health initiatives.

Another objective of the present disclosure is directed towards a system that features a simplified, intuitive interface, reducing barriers to healthcare access for populations with limited digital literacy.

Another objective of the present disclosure is directed towards offering a scalable healthcare solution that addresses inequities and allows underserved populations often excluded from traditional health initiatives.

Another objective of the present disclosure is directed towards encouraging for privacy-centric digital health solutions with a data-free operational model to ensure user trust for sensitive health topics like HIV and mental health.

Another objective of the present disclosure is directed towards promoting the integration of gamified tools in public health strategies to enhance engagement, particularly among younger populations.

Another objective of the present disclosure is directed towards supporting integrated healthcare approaches through government and organizational policies, moving away from fragmented care models to address interconnected health risks.

According to an exemplary aspect of the present disclosure, a first computing device comprising a processor configured to execute instructions from a preventative health screening module, wherein the preventative health screening module includes an intelligent virtual assistant (IVA) chatbot configured to interact with a user through a dynamic and AI-driven interface, whereby the intelligent virtual assistant (IVA) chatbot generates a series of intent-driven and personalized screening questions using Natural Language Processing (NLP) and Natural Language Understanding (NLU), thereby facilitating user engagement.

According to another exemplary aspect of the present disclosure, the intelligent virtual assistant (IVA) chatbot enables the user to respond to the screening questions by selecting predefined options, providing textual responses, and uploading images of oral and skin abnormalities for AI-based analysis, whereby the preventative health screening module collects the user responses and image data related to oral and skin abnormalities and transmits the collected data to a server over a network;

According to another exemplary aspect of the present disclosure, the server comprising a healthcare data processing module configured to receive and process the user responses and image data using AI-driven algorithms and a syndemic evaluation framework, wherein the syndemic evaluation framework evaluates the interplay between communicable diseases, including HIV, STIs, and Mpox, and non-communicable conditions, including diabetes, hypertension, and mental health disorders, whereby the healthcare data processing module generates personalized healthcare recommendations and numerical risk scores related to oral health, sexual health, mental health, and overall health using a proprietary scoring mechanism, and transmits the generated recommendations and risk scores to the first computing device;

According to another exemplary aspect of the present disclosure, the preventative health screening module on the first computing device displays the numerical risk scores and personalized healthcare recommendations to the user, and presents additional actionable options, including booking telehealth appointments, ordering home test kits, connecting with healthcare providers, and accessing rewards for health-promoting actions, whereby the preventative health screening module provides a gamification feature with a stigma meter, wherein the stigma meter visually represents the user's progress in overcoming stigma, dynamically updates based on user actions such as completing screenings, ordering test kits, and engaging with support resources, and provides rewards, including discounts, coupons, and gamified perks, thereby encouraging continued user engagement;

According to another exemplary aspect of the present disclosure, a second computing device and a third computing device communicatively connected to the server over the network, wherein the second computing device and the third computing device comprise the preventative health screening module configured to enable healthcare providers and case managers to securely access user-approved health data, including numerical risk scores, image analysis results, and personalized healthcare recommendations, whereby the second and third computing devices allow healthcare providers and case managers to make decisions and develop personalized care plans while maintaining user anonymity.

### BRIEF DESCRIPTION OF THE DRAWINGS

FIG. 1 is a block diagram depicting a schematic representation of an artificial intelligence-based system for automated preventative health screening and data-driven health risk analysis, in accordance with one or more exemplary embodiments.

FIG. 2 is a block diagram depicting an embodiment of the preventative health screening module 112 on the computing devices, in accordance with one or more exemplary embodiments.

FIG. 3 is a block diagram depicting an embodiment of the healthcare data processing module 114 on the server, in accordance with one or more exemplary embodiments.

FIG. 4 is a block diagram depicting an embodiment of the artificial intelligence-based system for automated preventative health screening and data-driven health risk analysis, in accordance with one or more exemplary embodiments.

FIG. 5A is a flow diagram depicting an artificial intelligence-based system for automated preventative health screening and data-driven health risk analysis, in accordance with one or more exemplary embodiments.

FIGS. 5B and 5C are flow diagrams depicting automated preventative health screening and data-driven health risk analysis, in accordance with one or more exemplary embodiments.

FIGS. 6A, 6B, 6C, and 6D are example diagrams depicting embodiments of the overall risk score, oral risk score mental risk score, and sexual risk score screens on the user interface module, in accordance with one or more exemplary embodiments.

FIG. 7 is a flow diagram depicting a method for generating oral risk score, in accordance with one or more exemplary embodiments.

FIG. 8 is a flow diagram depicting a method for generating a mental risk score, in accordance with one or more exemplary embodiments.

FIG. 9 is a flow diagram depicting a method for generating a sexual risk score, in accordance with one or more exemplary embodiments.

FIG. 10 is a flow diagram depicting a method for automated preventative health screenings, in accordance with one or more exemplary embodiments.

FIG. 11 is a flow diagram depicting a method for automated preventative health screening and data-driven health risk analysis, in accordance with one or more exemplary embodiments.

FIG. 12A to 12O are example screens depicting embodiments of the user interface module, in accordance with one or more exemplary embodiments.

FIG. 13A to 13P are example screens depicting embodiments of the user interface module that allow users to initiate and interact with various aspects of a preventative health screening, in accordance with one or more exemplary embodiments.

FIG. 14A is a schematic diagram illustrating a holistic model of an artificial intelligence-based system for automated preventative health screening and data-driven health risk analysis, in accordance with one or more exemplary embodiments.

FIG. 14B illustrates the syndemic screening approach employed by the preventative health screening module, in accordance with one or more exemplary embodiments.

FIG. 14C is an example diagram depicting the strong connection between oral, sexual, and mental health risks, in accordance with one or more exemplary embodiments.

FIG. 15A illustrates an overview of the interconnected preventative health screenings and functionalities provided by the system, including oral health analysis, sexual health analysis, mental health analysis, stigma reduction, and continuity of care.

FIG. 15B illustrates the stigma interplay across various health domains, including mental health stigma, HIV stigma, sexual health stigma, and oral health, and highlights how the system addresses these stigmas through user interactions.

FIG. 15C illustrates a flow diagram representing the study methods implemented for evaluating the system's platform features, participant demographics, risk analysis methods, and statistical analysis to assess the effectiveness of reducing stigma and improving healthcare access.

FIG. 15D illustrates a bar chart representing the impact of an intervention phase on user participation across two categories, namely HIV testing views and access to safe sex supplies, measured over three distinct phases.

FIG. 15E illustrates a series of graphical gauges representing the outcomes of user engagement with the automated preventative health screening and data-driven health risk analysis system, showing percentages for key areas of improvement, including awareness, stigma-free decision-making, access to safe sex supplies, and HIV testing assistance.

FIG. 16 is a block diagram illustrating the details of a digital processing system in which various aspects of the present disclosure are operative by the execution of appropriate software instructions.

### DETAILED DESCRIPTION OF EXAMPLE EMBODIMENTS

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and so forth, herein do not denote any order, quantity, or importance, but rather are used to distinguish one element from another.

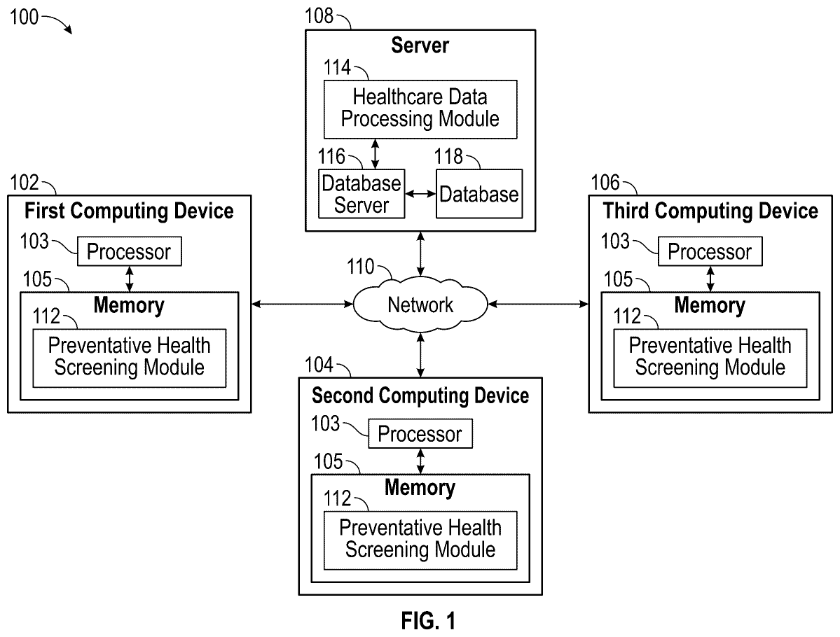

Referring to FIG. 1 is a block diagram depicting a schematic representation of an artificial intelligence-based system for automated preventative health screening and data-driven health risk analysis, in accordance with one or more exemplary embodiments. The system 100 includes a first computing device 102, a processor 103, a second computing device 104, a memory 105, a third computing device 106, a server 108, a network 110, a preventative health screening module 112, a healthcare data processing module 114, a database server 116, and a database 118.

The first computing device 102 may include a user device. The user may include but not limited to individuals, normal users, students, young people, teenagers, individuals living with HIV/STIs, and the like. The second computing device 104 may include a dental staff device. The third computing device 106 may include a primary care physician (PCP) device. The computing devices 102, 104, 106, may include but are not limited to, a personal digital assistant, smartphones, personal computers, mobile stations, computing tablets, handheld devices, an internet-enabled calling device, an internet enabled calling software, a telephone, a mobile phone, a digital processing system, and so forth. The computing devices 102, 104, and 106, may be communicatively connected to the server 108 over the network 110. The network 110 may include, but not limited to, an Internet of things (IoT network devices), an Ethernet, a wireless local area network (WLAN), or a wide area network (WAN), a Bluetooth low energy network, a ZigBee network, a WIFI communication network e.g., the wireless high speed internet, or a combination of networks, a cellular service such as a 4G (e.g., LTE, mobile WiMAX) or 5G cellular data service, a RFID module, a NFC module, wired cables, such as the world-wide-web based Internet, or other types of networks may include Transport Control Protocol/Internet Protocol (TCP/IP) or device addresses (e.g. network-based MAC addresses, or those provided in a proprietary networking protocol, such as Modbus TCP, or by using appropriate data feeds to obtain data from various web services, including retrieving XML data from an HTTP address, then traversing the XML for a particular node) and so forth without limiting the scope of the present disclosure.

Although the computing devices 102, 104, and 106 are shown in FIG. 1, an embodiment of the system 100 may support any number of computing devices. The computing devices 102, 104, and 106 may be operated by the users, dental staff, and primary care physician (PCP). The computing devices 102, 104, and 106 supported by the system 100 is realized as a computer-implemented or computer-based device having the hardware or firmware, software, and/or processing logic needed to carry out the computer-implemented methodologies described in more detail herein.

The computing devices 102, 104, and 106 may include the preventative health screening module 112. The preventative health screening module 112 may be any suitable applications downloaded from GOOGLE PLAY® (for Google Android devices), Apple Inc.'s APP STORE® (for Apple devices), or any other suitable database. The preventative health screening module 112 may be a desktop application which runs on Windows or Linux or any other operating system and may be downloaded from a webpage or a CD/USB stick etc. In some embodiments, the preventative health screening module 112 may be software, firmware, or hardware that is integrated into the computing devices 102, 104, 106. The computing devices 102, 104, and 106 may present a web page to the user by way of a browser, wherein the webpage comprises a hyper-link may direct the user to uniform resource locator (URL). In some embodiments, the preventative health screening module 112 may be an intelligent virtual assistance (IVA) chatbot.

The intelligent virtual assistance (IVA) chatbot may conduct automated sessions with a user. Herein, the session may refer to a time-continuous dialog between the intelligent virtual assistance (IVA) chatbot and the user. The dialog may include one or more questions related to the oral and mental sexual topics. The intelligent virtual assistance (IVA) chatbot allows the user to respond to the questions. The response may include any information input by the user, answers of the user to questions from the chatbot, opinions of the user, etc. The preventative health screening module 112 may guide the user through automated preventative health screening and data-driven health risk analysis, and the preventative health screening module 112 may gather the user inputs or responses. The preventative health screening module 112 may be configured to transfer the user inputs and responses to the server 108 over the network 110. The server 108 may include the healthcare data processing module 114. The healthcare data processing module 114 may be configured to receive the user inputs and responses from the preventative health screening module 112. The healthcare data processing module 114 may be configured to store the user inputs and responses on the database 118 through the database server 116. The healthcare data processing module 114 may be configured to analyze the information provided by the user, generate insights, perform risk analysis, and possibly integrate with healthcare databases or systems for additional analysis or referrals. The healthcare data processing module 114 may be configured to transfer the generated insights, and risk score to the preventative health screening module 112 over the network 110. The preventative health screening module 112 may be configured to receive the insights, and risk score from the healthcare data processing module 114. The preventative health screening module 112 may be configured to display the insights and the risk score to the user on the first computing device 102.

In accordance with one or more exemplary embodiments of the present disclosure, the first computing device 102 may include the processor 103 may be configured to execute instructions from the preventative health screening module 112. The preventative health screening module 112 may include an intelligent virtual assistant (IVA) chatbot may be configured to interact with a user through a dynamic and AI-driven interface. The intelligent virtual assistant (IVA) chatbot generates a series of intent-driven and personalized screening questions using Natural Language Processing (NLP) and Natural Language Understanding (NLU), thereby facilitating user engagement. The intelligent virtual assistant (IVA) chatbot enables the user to respond to the screening questions by selecting predefined options, providing textual responses, and uploading images of oral and skin abnormalities for AI-based analysis. The preventative health screening module 112 may collects the user responses and image data related to oral and skin abnormalities and transmits the collected data to a server over the network 110. The server 108 may include a healthcare data processing module may be configured to receive and process the user responses and image data using AI-driven algorithms and a syndemic evaluation framework. The syndemic evaluation framework evaluates the interplay between communicable diseases, including HIV, STIs, and Mpox, and non-communicable conditions, including diabetes, hypertension, and mental health disorders. The healthcare data processing module generates personalized healthcare recommendations and numerical risk scores related to oral health, sexual health, mental health, and overall health using a proprietary scoring mechanism, and transmits the generated recommendations and risk scores to the first computing device. The preventative health screening module 112 on the first computing device 102 may displays the numerical risk scores and personalized healthcare recommendations to the user, and presents additional actionable options, including booking telehealth appointments, ordering home test kits, connecting with healthcare providers, and accessing rewards for health-promoting actions. The preventative health screening module 112 provides a gamification feature with a stigma meter. The stigma meter visually represents the user's progress in overcoming stigma, dynamically updates based on user actions such as completing screenings, ordering test kits, and engaging with support resources, and provides rewards, including discounts, coupons, and gamified perks, thereby encouraging continued user engagement. The second computing device 104 and the third computing device 106 may be communicatively connected to the server over the network. The second computing device 104 and the third computing device 106 may include the preventative health screening module 112 may be configured to enable healthcare providers and case managers to securely access user-approved health data, including numerical risk scores, image analysis results, and personalized healthcare recommendations. The second and third computing devices 104 may allow healthcare providers and case managers to make decisions and develop personalized care plans while maintaining user anonymity.

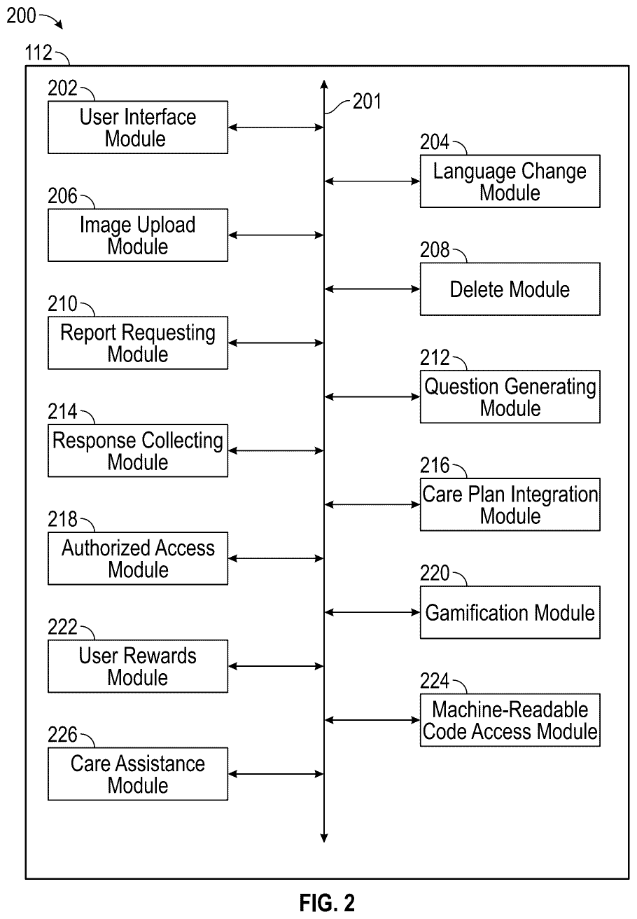

Referring to FIG. 2 is a block diagram 200 depicting an embodiment of the preventative health screening module 112 on the computing devices, in accordance with one or more exemplary embodiments. The preventative health screening module 112 includes a bus 201, a user interface module 202, a language change module 204, an image upload module 206, a delete module 208, a report requesting module 210, a question generating module 212, a response collecting module 214, a care plan integration module 216, and an authorized access module 218, a gamification module 220, a user rewards module 222, a machine-readable code access module 224, and a care assistance module 226. The bus 201 may include a path that permits communication among the modules of the preventative health screening module 112 installed on the computing devices 102, 104, and 106. The term “module” is used broadly herein and refers generally to a program resident in the memory of the computing devices 102, 104, and 106.

The user interface module 202 may be configured to enable the user to access the preventative health screening module 112 installed on the first computing device 102 as a guest without providing user details. The user interface module 202 may pop up a message to the user. The message may include for example: Thank you for your interest in “automated preventative health screening and data-driven health risk analysis”. You are one step away from taking charge of your overall sexual health awareness. This will take 2-3 mins of your time. DISCLAIMER: The result of this test will remain confidential and anonymous. Those will not be shared with anyone unless you wish to consult your provider. You also have the option to delete this chat once you receive your risk score. Retake the automated preventative health screening and data-driven health risk analysis anytime and anywhere. The user interface module 202 may offer three options to the user. The three options may include “Opt-In”, “Opt Out”, or “Know more”. The user interface module 202 may enable the user to select at least one option from the “Opt-In”, “Opt Out”, or “Know more” options. The user interface module 202 may be configured to redirect the user to the language change module 204 when the user selects the “Opt-In” option. The user interface module 202 may pop up a message when the user selects the “Opt Out” option. The message may include for example: Disclaimer: You are about to leave the automated preventative health screening. You can take the automated preventative health screening and data-driven health risk analysis anytime by visiting the link. The user interface module 202 may close the preventative health screening module 112.

The user interface module 202 may be configured to enable users to access the preventative health screening module 112 without requiring login credentials, ensuring complete anonymity and privacy. The user interface module 202 may allow users to interact with the system via multiple input modes, including text-based messages, voice inputs, or simple selection-based options, providing a user-friendly and intuitive experience. To encourage user engagement and reduce barriers to participation, the User Interface Module 202 may leverage oral health as a neutral and stigma-free entry point for initiating health screenings. By framing initial questions around oral health such as oral hygiene practices or identifying symptoms like mouth sores or gum issues the module creates a comfortable environment for users to begin their health analysis without directly addressing sensitive or stigmatized topics like sexual health, mental health, or substance use. This indirect approach builds trust and fosters user engagement, enabling seamless progression to broader interconnected risk analysis in a non-judgmental manner.

The user interface module 202 may further display options to guide users through the process, such as “Opt-In” to start the automated preventative health screening, “Opt-Out” to exit, or “Know More” to gather additional information about available services. Users opting in may be seamlessly redirected to multi-lingual support facilitated by the Language Change Module 204, ensuring inclusivity for diverse populations. The module may also present disclaimers emphasizing user confidentiality, such as privacy protections, anonymity of responses, and data deletion options. Additionally, the module may facilitate smooth navigation through dynamically generated screening questions, image uploads, and reward-based features, creating a cohesive user experience that balances trust, privacy, and accessibility.

The language change module 204 may be configured to enable users to select their preferred language for interacting with the preventative health screening module 112. This module serves as the user-facing interface, offering a seamless experience for individuals from diverse linguistic and cultural backgrounds. Upon user selection, the module may work in conjunction with the language translation module 318 on the server side to ensure real-time translation of all displayed text, prompts, and responses. The language change module 204 may provide users with culturally sensitive phrasing and intuitive design tailored to their selected language, ensuring comfort and inclusivity throughout their interaction. By addressing diverse linguistic needs, the language change module 204 fosters accessibility, allowing users to navigate and complete automated preventative health screening without language barriers.

The user interface module 202 may enable the user to input a text message or voice message in their own words own language to learn about HIV and Sexually Transmitted Diseases (STDs) when the user selects the “Know more” option. The text message or voice message may be like (1) Is there any preventative medicine after STI-exposed intercourse? (2) What are HIV/STD and oral health links? (3) What's PrEp (Pre exposure prophylaxis)? (4) Am I eligible and will my insurance cover the test/medicines? The user interface module 202 may display one or more options to the user based on the user-inputted text message and voice message. The option may include but not limited to Option 1: There is no surefire “preventative medicine” to eliminate the risk, but if think you have recently been exposed to HIV during sex (for example, if you had a condom break or you were sexually assaulted), talk to your health care provider or an emergency room doctor about PEP right away (within 72 hours). The sooner you start PEP, the better; every hour counts. For more information redirects the user to www.hiv.gov.in. Option 2: Oral symptoms are common for people with HIV, with anywhere from 30% to 80% of individuals experiencing some type of mouth sore or lesion on the tongue, or cheeks. For more information redirect the user to www.verywellhealth.com. Option 3: If you are at risk of HIV exposure through sex or injection drug use, ask a healthcare professional if PrEP is right for you. PrEP medications only protect against HIV. Option 4: Your eligibility depends on various factors. If you receive PrEP medication through the Ready, Set, PrEP program, you will not have to pay for the medicine, and the like. The language change module 204 may be configured to enable the user to change the communication language on the preventative health screening module 112. The language may include but not limited to English, Spanish, and the like. The question generating module 212 may be configured to generate questions related to oral health information, behavioral health information, sexual life information, drug usage information, and sexual abuse information. The generated question may be displayed on the user interface module 202. The image upload module 206 may be configured to allow the user to select an option for uploading the oral and skin abnormalities images. The user interface module 202 may be configured to allow the user to provide oral health information, behavioral health information, sexual life information, drug usage information, and sexual abuse information data.

The preventative health screening module 112 may be configured to transfer the user-provided oral health information, behavioral health information, sexual life information, drug usage information, and sexual abuse information data to the server 108 over network 110. The delete module 208 may be configured to enable the user to delete and close the chat. The delete module 208 may be configured to allow users to delete their chat histories, screening results, and uploaded images at any point. The delete module 208 ensures user privacy and complete control over their health data. The report requesting module 210 may be configured to enable the user to request the pdf file of the image risk score, oral risk score, mental risk score, sexual risk score, and overall risk score. The response collecting module 214 may be configured to collect oral health information, behavioral health information, sexual life information, drug usage information, and sexual abuse information response from the user. In accordance with one or more exemplary embodiments of the present disclosure, the preventative health screening module 112 may be configured to enable the user to order/gift a free home test kit for themself. The preventative health screening module 112 may be configured to enable the user to order/gift a free home test kit for someone else.

The question generating module 212 may be configured to dynamically generate and adapt screening questions using advanced Natural Language Processing (NLP) and Natural Language Understanding (NLU) algorithms. The question generating module 212 leverages real-time analysis of user inputs to personalize the screening experience, ensuring that questions are intent-driven and directly relevant to the user's responses. By analyzing both explicit and implicit user inputs, the module identifies the underlying intent and tailors subsequent questions to address specific health domains, such as oral health, sexual health, behavioral health, substance use, and mental health. For example, if a user mentions concerns related to oral hygiene, the system may adapt by presenting follow-up questions that assess broader health risks while maintaining a non-intrusive approach.

The question generating module 212 optimizes the sequence and phrasing of questions to balance user comfort and screening accuracy. This ensures that the flow of questions progresses logically and intuitively, fostering a smooth and engaging automated preventative health screening process. Through its adaptive nature, the module enables more accurate evaluations of health risks by dynamically responding to user inputs in real-time, making the system highly personalized and efficient across diverse health domains. The module's ability to align the questions with user intent not only improves the precision of risk analysis but also enhances user engagement by creating a conversational and supportive interaction environment.

The image upload module 206 may be configured to enable users to upload images of oral and skin abnormalities, such as lesions, discoloration, or other visible conditions, as part of the automated preventative health screening process. To ensure clarity and usability, the module may provide users with step-by-step instructions or guidance to capture high-quality and appropriate images for analysis. These instructions may include recommendations for lighting, focus, and positioning to enhance the accuracy of the captured visual data. Once the images are uploaded, the image upload module 206 facilitates their secure transfer to the image processing and analysis module 314 located on the server side for further AI-driven evaluation. The module ensures that the user experience remains seamless, anonymous, and privacy-focused during the image submission process.

The care plan integration module 216 may be configured to generate personalized care plans for healthcare providers such as case managers, providers, and others. This integration helps them make timely and informed decisions, improving the quality of care for users. The care plan integration module 216 may work by integrating the chatbot with the preventative health screening module to automatically generate personalized care plans for healthcare providers like case managers, doctors, and others. The personalized care plans guide healthcare providers in making timely and informed decisions, ultimately improving the user's quality of care. The integration ensures that healthcare professionals have access to all the relevant data they need to manage the user's care effectively.

The care plan integration module 216 may be configured to generate personalized and actionable healthcare recommendations for users based on their screening results, risk scores, and AI-driven analyses. The care plan integration module 216 identifies key health needs and guides users toward appropriate healthcare pathways, such as eligibility checks for preventive medications like PrEP (pre-exposure prophylaxis), PEP (post-exposure prophylaxis), and Naloxone. It may also provide tailored suggestions for medication adherence tools, ensuring users can maintain consistency with prescribed treatments. Additionally, the care plan integration module 216 may direct users to guided testing workflows, enabling them to seamlessly order at-home test kits or book telehealth consultations. Users may also be connected to peer support networks, fostering a community-driven approach to healthcare. By integrating these recommendations into the user journey, this module ensures a cohesive and supportive experience that simplifies healthcare access and encourages proactive health management. The personalized care plans generated by the module empower users to take informed steps toward improving their overall health and well-being. The care plan integration module 216 may be configured to generate personalized care recommendations for users based on their screening results and risk scores. It may guide users toward actionable healthcare pathways, such as booking telehealth appointments, accessing test kits, or receiving medication adherence tools.

The authorized access module 218 may be configured to provide a complete view of a user's health data for case managers (educators, disease intervention specialists, infectious disease experts), providers (doctors, dentists, nurses), and others, enabling them to manage and make informed decisions based on comprehensive screening results. The authorized access module 218 may be configured to enable the case managers, providers, and others must verify their profiles through their organization before accessing the preventative health screening module, ensuring that only authorized professionals may use the preventative health screening module for case management and healthcare decisions. The authorized access module 218 ensures that only verified and authorized healthcare professionals can access the user's sensitive health data and the generated care plans. Before gaining access, case managers, doctors, and other providers must verify their credentials through their organizations. This security measure is critical to protecting user privacy and ensuring that only trusted individuals with valid professional credentials can use the system for case management and healthcare decisions.

The gamification module 220 may be configured to engage and motivate users through interactive gamification features, including a dynamic tool called the “Stigma Meter.” The Stigma Meter visually represents a user's progress in overcoming health-related stigma by tracking specific actions, such as completing health screenings, uploading images for analysis, ordering at-home test kits, accessing care resources, or engaging with peer support tools. As users complete these actions, the meter adjusts dynamically, providing real-time feedback and reflecting their advancement. The module may reward users for their progress with tangible incentives, such as coupons, discounts, or other gamified perks, to encourage proactive health behaviors. By combining visual progress indicators with rewards, the module fosters a sense of accomplishment, reduces the psychological barriers associated with stigmatized health topics like HIV or mental health, and promotes sustained user engagement. This approach transforms health management into a more approachable and empowering experience, helping users build confidence in addressing their health needs while normalizing stigma-free conversations. The gamification module 220 ensures that user interactions remain supportive, judgment-free, and engaging by integrating elements of positive reinforcement, progress tracking, and reward-driven motivation. The user rewards module 222 may be configured to monitor user progress and actions within the system. The user rewards module 222 may be configured to issue rewards, such as incentives, coupons, and discounts, for completing screenings or engaging with recommended healthcare services.

The machine-readable code access module 224 may be configured to enable users to access the preventative health screening module via QR codes. This feature allows for seamless entry into the platform without requiring login credentials, ensuring anonymity and ease of access. The care assistance module 226 may be configured to provide users with reminders, guided workflows, and eligibility forms for healthcare services. It may streamline the user's care journey by simplifying processes such as follow-up actions, guided testing, and prescription adherence.

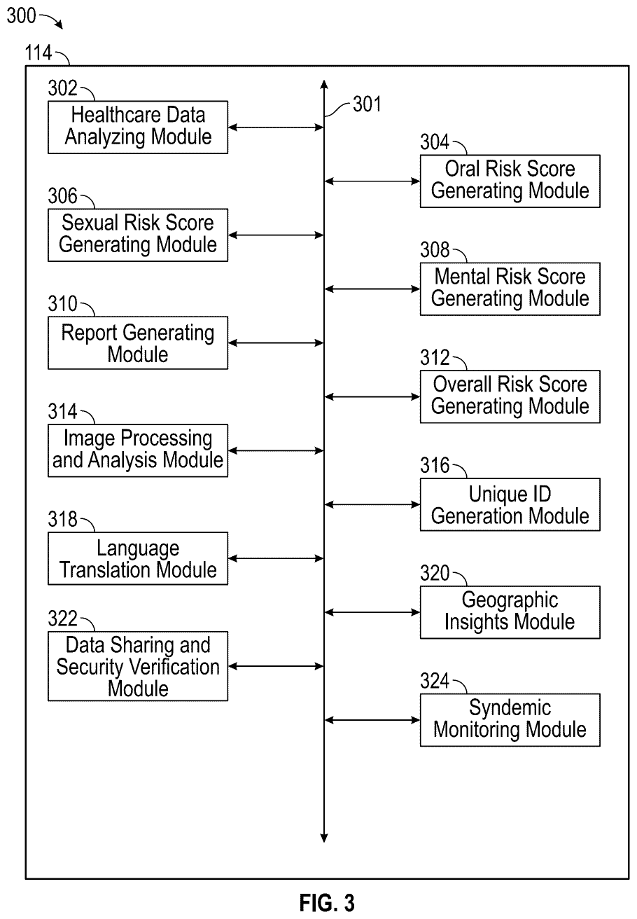

Referring to FIG. 3 is a block diagram 300 depicting an embodiment of the healthcare data processing module 114 on the server, in accordance with one or more exemplary embodiments. The healthcare data processing module 114 includes a bus 301, a healthcare data analyzing module 302, an oral risk score generating module 304, a sexual risk score generating module 306, a mental risk score generating module 308, a report generating module 310, and an overall risk score generating module 312, an image processing and analysis module 314, a unique id generation module 316, a language translation module 318, a geographic insights module 320, a data sharing and security verification module 322, and a syndemic monitoring module 324. The bus 301 may include a path that permits communication among the modules of the healthcare data processing module 114 on the server. The term “module” is used broadly herein and refers generally to a program resident in the server.

The healthcare data analyzing module 302 may be configured to process and analyze oral health information, behavioral health information, sexual life information, drug usage information, sexual abuse information, and image data of oral and skin abnormalities and assign numerical values to the health data. The oral risk score generating module 304 may be configured to generate the oral risk score of the user based on the processed data. The mental risk score generating module 308 may be configured to generate the mental risk score of the user based on the processed data. The sexual risk score generating module 306 may be configured to generate the sexual risk score of the user based on the processed data. The overall risk score generating module 312 may be configured to generate the overall risk score based on the processed data. The report generating module 310 may be configured to generate the pdf file with details of image risk score, oral risk score, mental risk score, sexual risk score, and overall risk score. The report generating module 310 may also be configured to generate comprehensive, downloadable reports containing user risk scores, AI analysis results, and care recommendations.

The healthcare data analyzing module 302 may be configured to process and analyze a wide range of user-provided health data, including questionnaire responses, behavioral inputs, and uploaded images of oral and skin abnormalities. The healthcare data analyzing module 302 integrates advanced Artificial Intelligence (AI) algorithms with a unique syndemic evaluation framework to assess the complex interplay between multiple health conditions. Specifically, it evaluates the interconnected risks posed by communicable diseases, such as HIV, STIs, and Mpox, alongside non-communicable conditions like diabetes, hypertension, and mental health disorders. By leveraging this syndemic framework, the healthcare data analyzing module 302 identifies correlations between systemic health risks and evaluates how one condition may exacerbate or influence another. For instance, oral health abnormalities linked to immunosuppression may be cross-referenced with mental health stressors and behavioral risk factors to provide a comprehensive and nuanced risk profile. This integrated analysis empowers both users and healthcare providers to better understand the interconnected nature of health risks, offering a holistic view of the user's overall health status. The outputs of this module form the foundation for generating personalized risk scores and actionable recommendations, ensuring that users receive precise, targeted insights into their health. By addressing both individual and systemic health risks, the healthcare data analyzing module 302 supports informed decision-making and encourages proactive healthcare engagement, bridging gaps between traditionally siloed healthcare domains.

The report generating module 310 may be configured to produce comprehensive, downloadable reports that consolidate user risk scores, AI analysis results, and care recommendations generated by the automated preventative health screening and data-driven health risk analysis system. These reports may include detailed insights tailored to the user's unique health profile, offering actionable pathways such as booking telehealth appointments, ordering test kits, or accessing peer support services. The module ensures that all recommendations provided in the reports are actionable and easy to understand, helping users navigate complex healthcare decisions. By integrating results from various modules, including risk analysis and eligibility checks for medications, the report generating module 310 facilitates seamless care linkage and ensures users are equipped with all necessary information to proceed confidently in their healthcare journey. The generated reports also serve as a valuable resource for healthcare providers, enabling them to deliver more targeted and efficient care.

The image processing and analysis module 314 may be configured to analyze user-uploaded images of oral and skin abnormalities using advanced AI-based computer vision algorithms. The image processing and analysis module 314 processes the visual data to detect, identify, and classify potential health conditions, such as oral lesions, gum diseases, or skin irregularities, which may indicate broader systemic risks, including HIV, HPV, or other health issues. By integrating objective image analysis with subjective user inputs provided during the analysis, the image processing and analysis module 314 enhances the accuracy of health risk evaluations. The analyzed results, which may include identified abnormalities and risk indicators, are transmitted to the risk score generating modules 304, 306, 308, 312. These modules incorporate the visual analysis data into the calculation of personalized risk scores, such as oral health risk scores, mental health risk scores, and overall health risk scores. This unique combination of AI-driven image analysis and user-provided data ensures a comprehensive, multi-dimensional health analysis, delivering precise and actionable insights for users.

The unique id generation module 316 may be configured to generate a secure, unique alphanumeric identifier for each user upon completing a screening within the automated preventative health screening and data-driven health risk analysis system. This identifier ensures complete anonymity by allowing users to interact with the platform without revealing any personally identifiable information. The unique ID enables users to securely perform various follow-up actions, including ordering at-home test kits, scheduling telehealth consultations, accessing personalized health reports, and claiming rewards or incentives through the system. The module facilitates seamless engagement with healthcare services by acting as a secure and private reference key for each user, ensuring continuity without requiring login credentials or additional identity verification. This functionality not only enhances user trust by prioritizing anonymity but also maintains critical system functionalities, such as linking screening results to follow-up actions and rewards. By providing a robust mechanism for secure and anonymous engagement, the Unique ID Generation Module 316 supports users in accessing essential healthcare resources while ensuring privacy and data security.

The risk score generating modules 304, 306, 308, and 312 may be configured to compute personalized health risk scores through a proprietary scoring mechanism, termed the “Chance Screening Scores.” These scores are designed to classify health risks into distinct categories, including oral health, sexual health, mental health, and overall health. The oral risk score generating module 304 may compute risk scores based on oral health data, including AI-driven analysis of uploaded images of oral abnormalities such as lesions, gum conditions, or discolorations. The sexual risk score generating module 306 may assess risks based on user-provided inputs regarding sexual health behaviors, symptoms, and histories. The mental risk score generating module 308 may calculate mental health risk levels using responses to behavioral and emotional well-being queries. The overall risk score generating module 312 may integrate the outputs from the individual modules, combining user responses, AI-driven image analysis, and relevant health data into a comprehensive, multi-dimensional evaluation. This framework ensures a balanced analysis by combining both subjective inputs (e.g., user responses) and objective analysis (e.g., image-based data and AI algorithms). The resulting Chance Screening Scores provide actionable insights to users and healthcare providers, enabling early detection of systemic health risks and supporting informed decision-making. By categorizing risks across interconnected health domains, the Risk Score Generating Modules contribute to a holistic understanding of the user's health status.

The language translation module 318 may be configured to perform real-time language translations for user interactions, ensuring the accuracy and cultural relevance of communication. The language translation module 318 leverages advanced AI-based translation algorithms to dynamically translate both static and user-inputted content across multiple languages. This ensures that users can engage with the system naturally and confidently in their preferred language while maintaining the integrity of healthcare-related information. Additionally, the language translation module 318 ensures that all translated communication adheres to anonymous data handling protocols, safeguarding user privacy and ensuring that no personal information is compromised during the translation process. By combining real-time accessibility with culturally sensitive content, the module promotes inclusivity for diverse populations, encouraging broader participation in the automated preventative health screening. The geographic insights module 320 may be configured to provide anonymized, location-based health insights by aggregating user data. The data sharing and security verification module 322 may be configured to ensure secure access to user-approved data by verified healthcare providers. The syndemic monitoring module 324 may be configured to analyze aggregated health data to identify syndemic patterns and health trends for public health monitoring.

In accordance with one or more exemplary embodiments of the present disclosure, the preventative health screening module 112 may pop up a message if the oral risk score is >65% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label Consult Immediately. The preventative health screening module 112 may pop up a message if the oral risk score is >65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label Consult Immediately.

In accordance with one or more exemplary embodiments of the present disclosure, the preventative health screening module 112 may pop up a message if the oral risk score is 30-65% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention. The preventative health screening module 112 may pop up a message if the oral risk score is 30-65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention. The preventative health screening module 112 may pop up a message if the oral risk score is <30% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention. The preventative health screening module 112 may pop up a message if the oral risk score is <30% and the image classification output is 0 the message may include based on the responses you have provided, it seems like there may be a “Low chance” to the lesions in the image you uploaded. For better clarity and understanding, it's advisable to consult with a peer educator or consider taking a test. The preventative health screening module 112 may display the label safe.

In accordance with one or more exemplary embodiments of the present disclosure, the preventative health screening module 112 may pop up a message if the sexual risk score is >65% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label consult immediately. The preventative health screening module 112 may pop up a message if the sexual risk score is >65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label consult immediately. The preventative health screening module 112 may pop up a message if the sexual risk score is 30-65% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention. The preventative health screening module 112 may pop up a message if the sexual risk score is 30-65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention.

In accordance with one or more exemplary embodiments of the present disclosure, the preventative health screening module 112 may pop up a message if the sexual risk score is <30% and the image classification output is 1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the label that needs attention. The preventative health screening module 112 may pop up a message if the sexual risk score is <30% and the image classification output is 0 the message may include based on the responses you have provided, it seems like there may be a Low Chance of the lesions in the image you uploaded. For better clarity and understanding, it's advisable to consult with a peer educator or consider taking a test. The preventative health screening module 112 may display the safe label.

In accordance with one or more exemplary embodiments of the present disclosure, the preventative health screening module 112 may pop up a message if the overall risk score is >65% and the image classification output is >=1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the consult immediately label. The preventative health screening module 112 may pop up a message if the overall risk score is >65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the consult immediately label.

In accordance with one or more exemplary embodiments of the present disclosure, the preventative health screening module 112 may pop up a message if the overall risk score is 30-65% and the image classification output is 2 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the consult immediately label. The preventative health screening module 112 may pop up a message if the overall risk score is 30-65% and the image classification output is >=1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the needs attention label. The preventative health screening module 112 may pop up a message if the overall risk score is 30-65% and the image classification output is 0 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the needs attention label.

In accordance with one or more exemplary embodiments of the present disclosure, the preventative health screening module 112 may pop up a message if the overall risk score is <30% and the image classification output is 2 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the needs attention label. The preventative health screening module 112 may pop up a message if the overall risk score is <30% and the image classification output is >=1 the message may include based on your response and the overall analysis, it appears that there may be a “High Chance” to the lesions in the image you submitted. You may require assistance; for better clarity and understanding, consult with a peer educator or consider taking a test. The preventative health screening module 112 may display the needs attention label. The preventative health screening module 112 may pop up a message if the overall risk score is <30% and the image classification output is 0 the message may include based on the responses you have provided, it seems like there may be a Low Chance to the lesions in the image you uploaded. For better clarity and understanding, it's advisable to consult with a peer educator or consider taking a test. The preventative health screening module 112 may display the safe label.

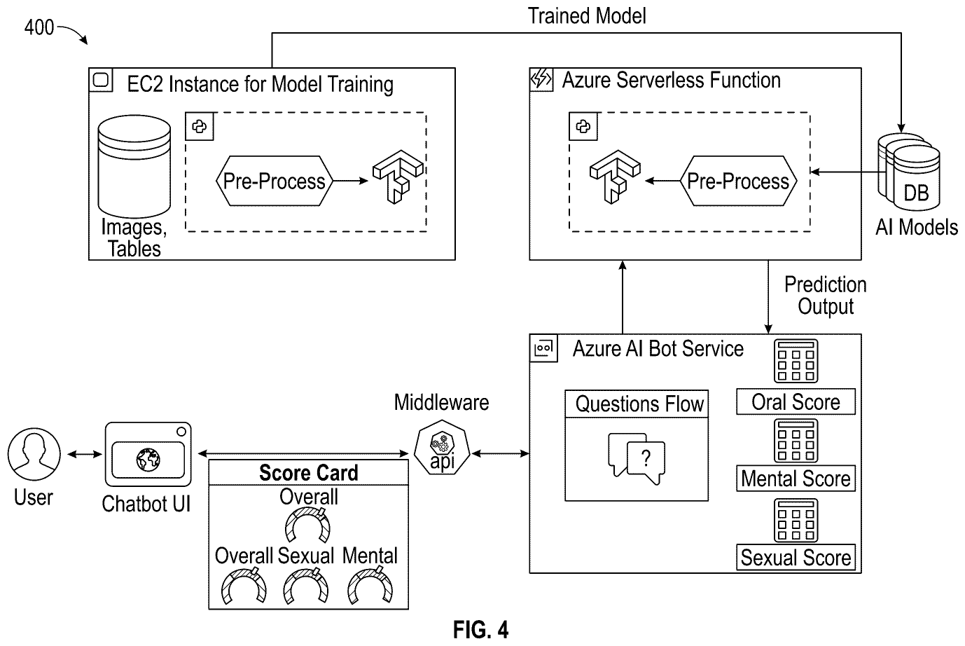

Referring to FIG. 4 is a block diagram depicting an embodiment of the artificial intelligence-based system for automated preventative health screening and data-driven health risk analysis, in accordance with one or more exemplary embodiments. The intelligent virtual assistance (IVA) chatbot may conduct automated sessions with a user. Herein, the session may refer to a time-continuous dialog between the intelligent virtual assistance (IVA) chatbot and the user. The dialog may include one or more questions related to the oral and mental sexual topics. The intelligent virtual assistance (IVA) chatbot allows the user to respond to the questions. The response may include any information input by the user, answers of the user to questions from the chatbot, opinions of the user, etc. The preventative health screening module 112 may guide the user through automated preventative health screening, and the preventative health screening module 112 may gather the user inputs or responses. The preventative health screening module 112 may be configured to transfer the user inputs and responses to the server 108 over the network 110. The server 108 may include the healthcare data processing module 114. The healthcare data processing module 114 may be configured to receive the user inputs and responses from the preventative health screening module 112. The healthcare data processing module 114 may be configured to store the user inputs and responses on the database 118 through the database server 116. The healthcare data processing module 114 may be configured to analyze the information provided by the user, generate insights, perform risk analysis, and possibly integrate with healthcare databases or systems for additional analysis or referrals. The healthcare data processing module 114 may be configured to transfer the generated insights, and risk score to the preventative health screening module 112 over the network 110. The preventative health screening module 112 may be configured to receive the insights, and risk score from the preventative health screening module 112. The preventative health screening module 112 may be configured to display the insights and the risk score to the user on the first computing device 102. The preventative health screening module 112 may be configured to transfer the image risk score, oral risk score, mental risk score, sexual risk score, and overall risk score to the second computing device 104, and the third computing device 104.

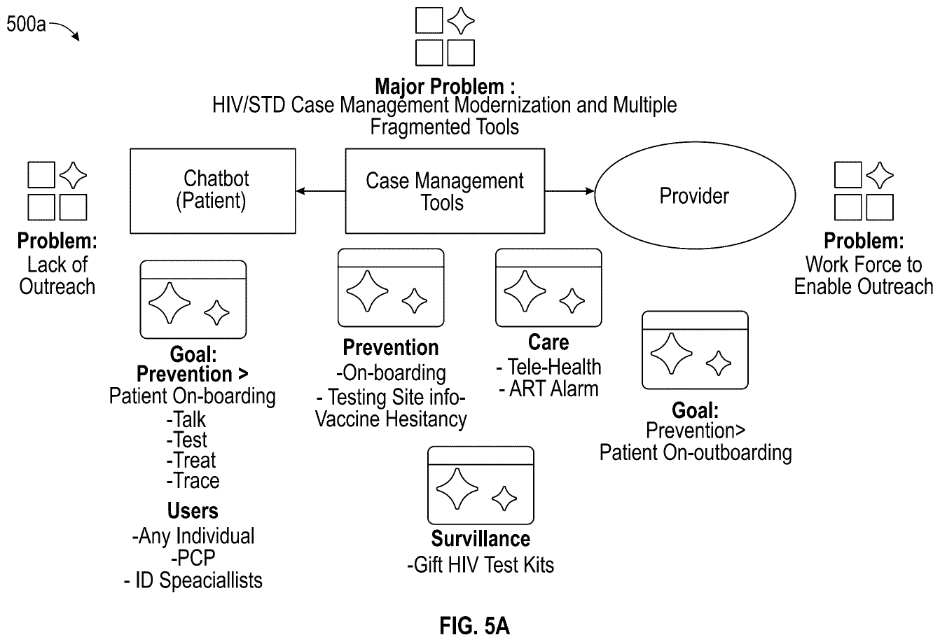

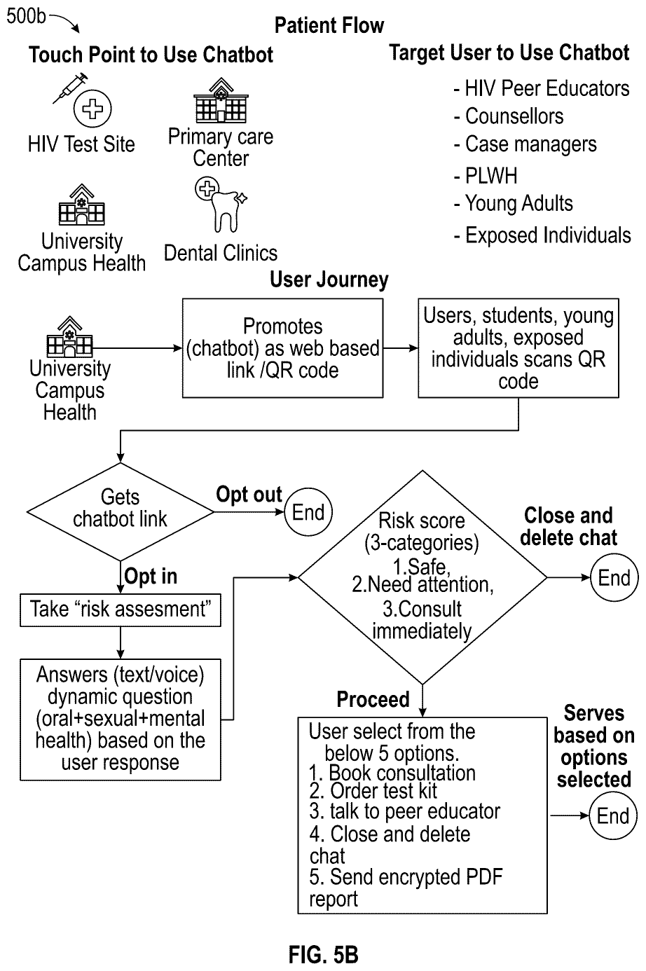

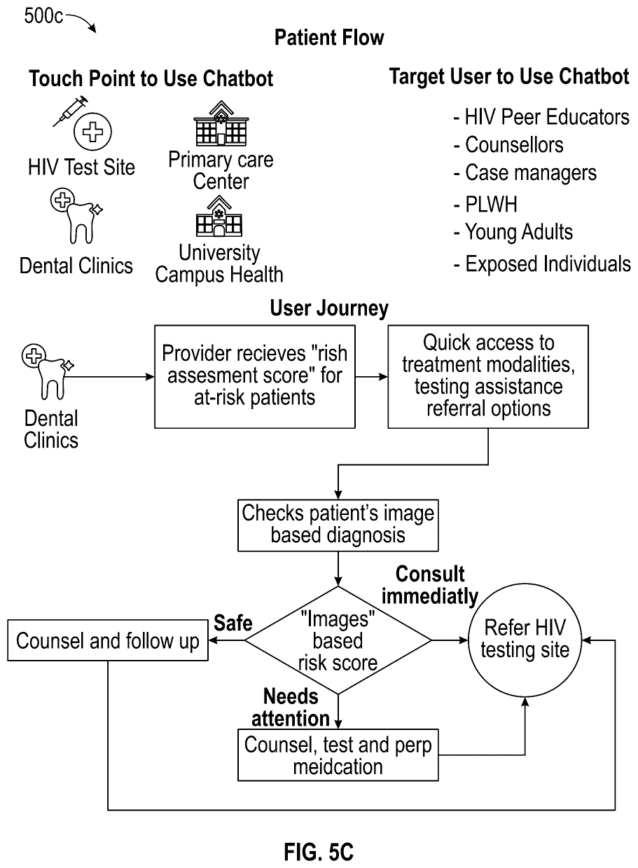

Referring to FIG. 5A is a flow diagram depicting an artificial intelligence-based system for automated preventative health screening and data-driven health risk analysis, in accordance with one or more exemplary embodiments. The intelligent virtual assistance (IVA) chatbot may conduct automated sessions with a user. Herein, the session may refer to a time-continuous dialog between the intelligent virtual assistance (IVA) chatbot and the user. The dialog may include one or more questions related to the oral and mental sexual topics. The intelligent virtual assistance (IVA) chatbot allows the user to respond to the questions. The response may include any information input by the user, answers of the user to questions from the chatbot, opinions of the user, etc. The preventative health screening module 112 may guide the user through the automated preventative health screening, and the preventative health screening module 112 may gather the user inputs or responses. The preventative health screening module 112 may be configured to transfer the user inputs and responses to the server 108 over the network. The server 108 may include the healthcare data processing module 114. The healthcare data processing module 114 may be configured to receive the user inputs and responses from the preventative health screening module 112. The healthcare data processing module 114 may be configured to store the user inputs and responses on the database 118 through the database server 116. The healthcare data processing module 114 may be configured to analyze the information provided by the user, generate insights, perform risk analysis, and possibly integrate with healthcare databases or systems for additional analysis or referrals. The healthcare data processing module 114 may be configured to transfer the generated insights, and risk score to the preventative health screening module 112 over the network 110. The preventative health screening module 112 may be configured to receive the insights, and risk score from the preventative health screening module 112. The preventative health screening module 112 may be configured to display the insights and the risk score to the user on the first computing device 102. The preventative health screening module 112 may be configured to transfer the image risk score, oral risk score, mental risk score, sexual risk score, and overall risk score to the second computing device 104, and the third computing device 104.

Referring to FIGS. 5B and 5C are flow diagrams depicting automated preventative health screening and data-driven health risk analysis, in accordance with one or more exemplary embodiments. The intelligent virtual assistance (IVA) chatbot may conduct automated sessions with a user. Herein, the session may refer to a time-continuous dialog between the intelligent virtual assistance (IVA) chatbot and the user. The dialog may include one or more questions related to the oral and mental sexual topics. The intelligent virtual assistance (IVA) chatbot allows the user to respond to the questions. The response may include any information input by the user, answers of the user to questions from the chatbot, opinions of the user, etc. The preventative health screening module 112 may guide the user through the automated preventative health screening, and the preventative health screening module 112 may gather the user inputs or responses. The preventative health screening module 112 may be configured to transfer the user inputs and responses to the server 108 over the network. The server 108 may include the healthcare data processing module 114. The healthcare data processing module 114 may be configured to receive the user inputs and responses from the preventative health screening module 112. The healthcare data processing module 114 may be configured to store the user inputs and responses on the database 118 through the database server 116. The healthcare data processing module 114 may be configured to analyze the information provided by the user, generate insights, perform risk analysis, and possibly integrate with healthcare databases or systems for additional analysis or referrals. The healthcare data processing module 114 may be configured to transfer the generated insights, and risk score to the preventative health screening module 112 over the network 110. The preventative health screening module 112 may be configured to receive the insights, and risk score from the preventative health screening module 112. The preventative health screening module 112 may be configured to display the insights and the risk score to the user on the first computing device 102. The preventative health screening module 112 may be configured to transfer the image risk score, oral risk score, mental risk score, sexual risk score, and overall risk score to the second computing device 104, and the third computing device 104.

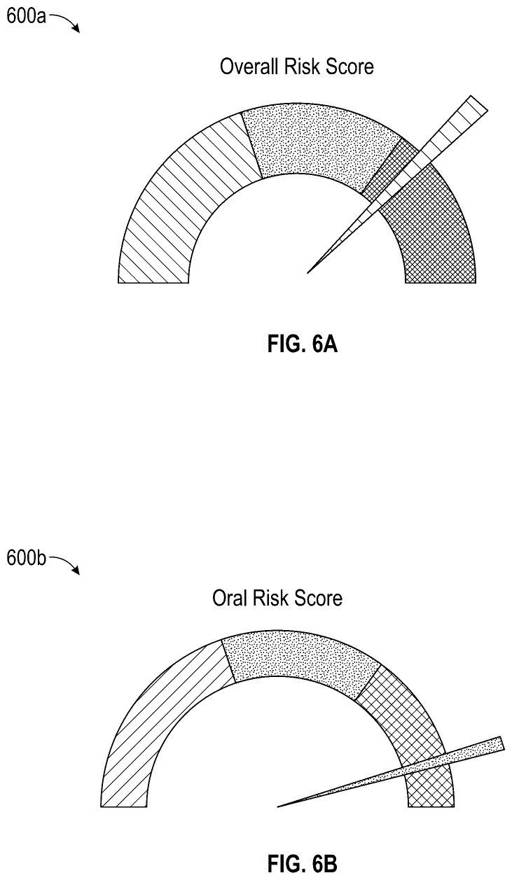

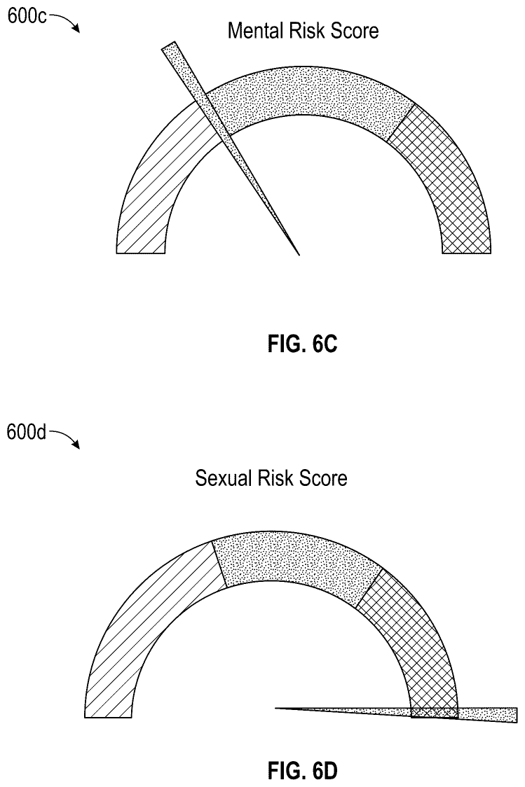

Referring to FIGS. 6A, 6B, 6C, and 6D are example diagrams 600a, 600b, 600c, and 600d depicting embodiments of the overall score, oral score mental score, and sexual score screens on the user interface module, in accordance with one or more exemplary embodiments.

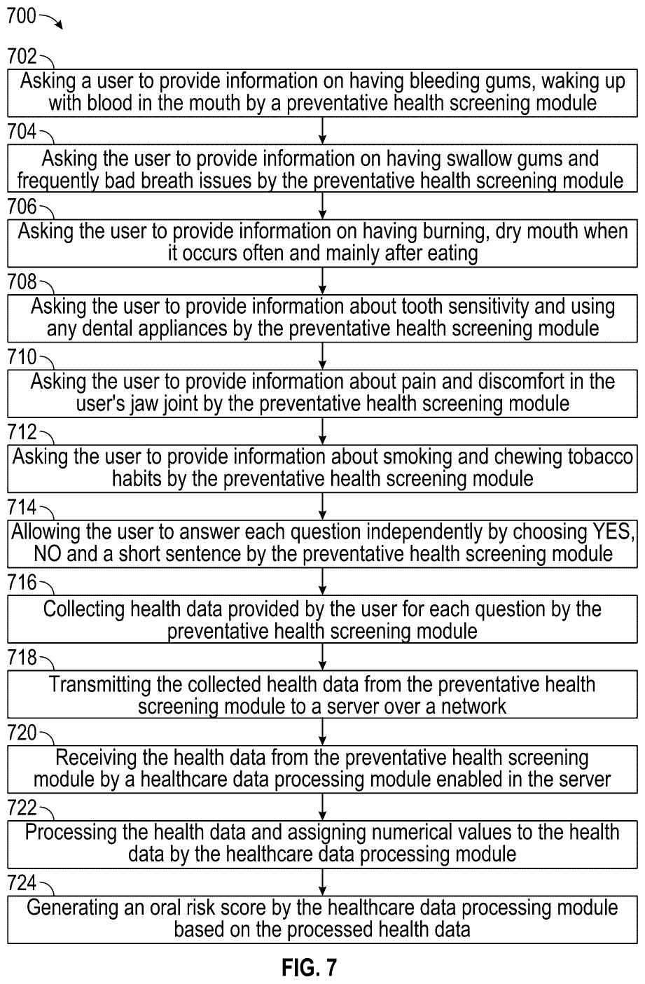

Referring to FIG. 7 is a flow diagram 700 depicting a method for generating oral risk score, in accordance with one or more exemplary embodiments. The method 700 may be carried out in the context of the details of FIG. 1, FIG. 2, FIG. 3, FIG. 4, FIG. 5A, FIG. 5B, FIG. 5C, FIG. 6A, FIG. 6B, FIG. 6C, and FIG. 6D. However, the method 700 may also be carried out in any desired environment. Further, the aforementioned definitions may equally apply to the description below.

The exemplary method 700 commences at step 702, asking a user to provide information on having bleeding gums and waking up with blood in his/her mouth by a preventative health screening module. Thereafter at step 704, asking the user to provide information on having swallow gums and frequently bad breath issues by the preventative health screening module. Thereafter at step 706, asking the user to provide information on having burning and dry mouth and when it occurs often and mainly after eating by the preventative health screening module. Thereafter at step 708, asking the user to provide information about tooth sensitivity and using any dental appliances (e.g., braces, dentures, bridges) by the preventative health screening module. Thereafter at step 710, asking the user to provide information about pain and discomfort in the user's jaw joint by the preventative health screening module. Thereafter at step 712, asking the user to provide information about smoking and chewing tobacco habits by the preventative health screening module. Thereafter at step 714, allowing the user to answer each question independently by choosing YES, or NO and providing a short sentence through the preventative health screening module. Thereafter at step 716, collecting health data provided by the user for each question by the preventative health screening module. Thereafter at step 718, transmitting the collected health data from the preventative health screening module to a server over a network. Thereafter at step 720, receiving the health data from the preventative health screening module by a healthcare data processing module enabled in server. Thereafter at step 722, processing the health data and assigning numerical values to the health data by the healthcare data processing module. Thereafter at step 724, generating an oral risk score by the healthcare data processing module based on the processed health data.

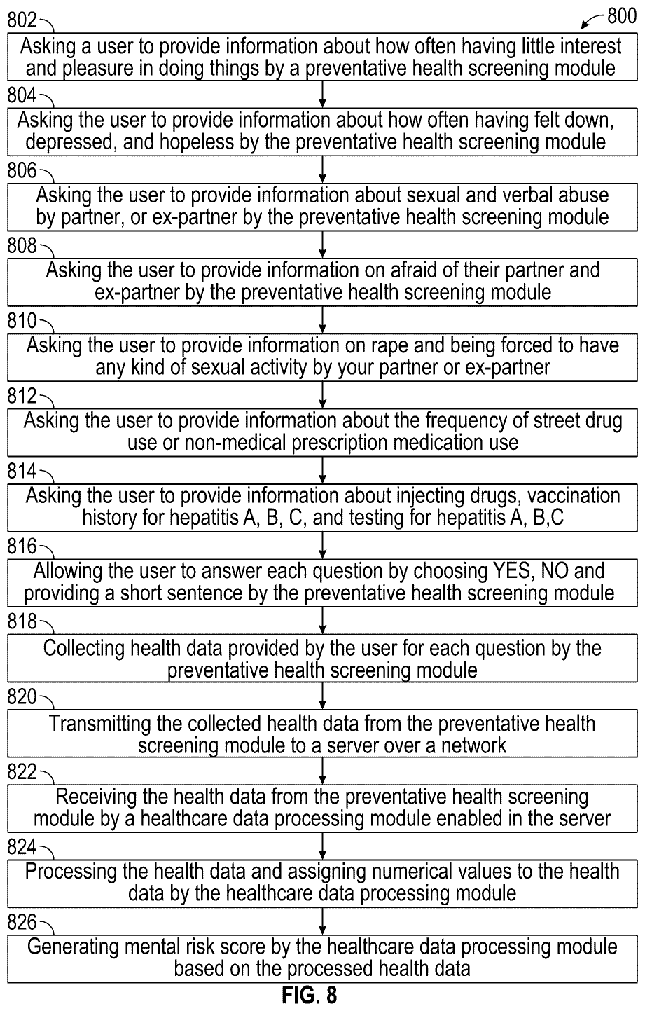

Referring to FIG. 8 is a flow diagram 800 depicting a method for generating a mental risk score, in accordance with one or more exemplary embodiments. The method 800 may be carried out in the context of the details of FIG. 1, FIG. 2, FIG. 3, FIG. 4, FIG. 5A, FIG. 5B, FIG. 5C, FIG. 6A, FIG. 6B, FIG. 6C, FIG. 6D, and FIG. 7. However, the method 800 may also be carried out in any desired environment. Further, the aforementioned definitions may equally apply to the description below.

The exemplary method 800 commences at step 802, asking a user to provide information about how often having little interest and pleasure in doing things by a preventative health screening module. Thereafter at step 804, asking a user to provide information about how often having felt down, depressed, and hopeless by a preventative health screening module. Thereafter at step 806, asking a user to provide information about experiencing sexual and verbal abuse by a partner, or ex-partner within the last year by the preventative health screening module. Thereafter at step 808, asking the user to provide information on afraid of their partner and ex-partner by the preventative health screening module. Thereafter at step 810, asking the user to provide information on rape and being forced to have any kind of sexual activity by his partner or ex-partner by the preventative health screening module. Thereafter at step 812, asking the user to provide information about the frequency of street (recreational) drug use or non-medical prescription medication use in the past year by the preventative health screening module. Thereafter at step 814, asking the user to provide information about injecting drugs, vaccination history for hepatitis A, B, C, and testing for hepatitis A, B, C. Thereafter at step 816, allowing the user to answer each question independently by choosing YES, NO and providing a short sentence through the preventative health screening module. Thereafter at step 818, collecting health data provided by the user for each question by the preventative health screening module. Thereafter at step 820, transmitting the collected health data from the preventative health screening module to a server over a network. Thereafter at step 822, receiving the health data from the preventative health screening module by a healthcare data processing module enabled in server. Thereafter at step 824, processing the health data and assigning numerical values to the health data by the healthcare data processing module. Thereafter at step 826, generating mental risk score by the healthcare data processing module based on the processed health data.

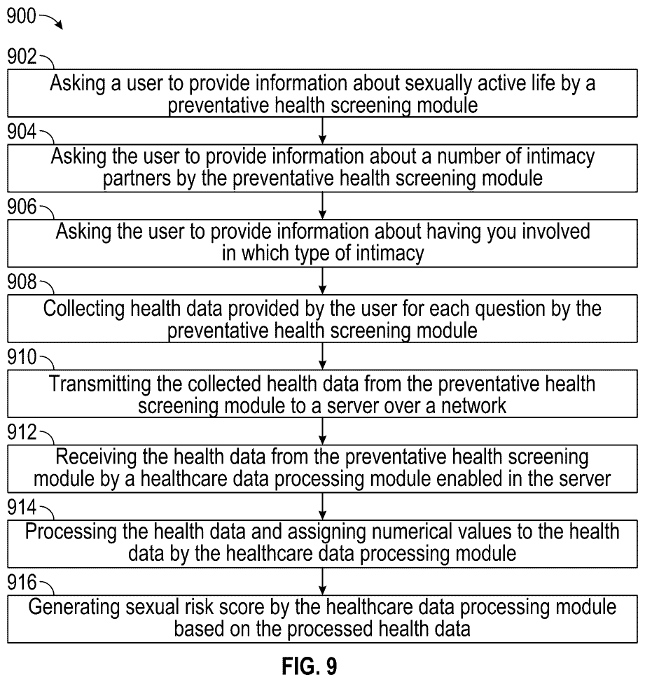

Referring to FIG. 9 is a flow diagram 900 depicting a method for generating a sexual risk score, in accordance with one or more exemplary embodiments. The method 900 may be carried out in the context of the details of FIG. 1, FIG. 2, FIG. 3, FIG. 4, FIG. 5A, FIG. 5B, FIG. 5C, FIG. 6A, FIG. 6B, FIG. 6C, FIG. 6D, FIG. 7, and FIG. 8. However, the method 900 may also be carried out in any desired environment. Further, the aforementioned definitions may equally apply to the description below.

The exemplary method 900 commences at step 902, asking a user to provide information about sexually active life by a preventative health screening module. Thereafter at step 904, asking a user to provide information about a number of intimacy partners by the preventative health screening module. Thereafter at step 906, asking a user to provide information about having user involved in which type of intimacy. Thereafter at step 908, collecting health data provided by the user for each question by the preventative health screening module. Thereafter at step 910, transmitting the collected health data from the preventative health screening module to a server over a network. Thereafter at step 912, receiving the health data from the preventative health screening module by a healthcare data processing module enabled in server. Thereafter at step 914, processing the health data and assigning numerical values to the health data by the healthcare data processing module. Thereafter at step 916, generating sexual risk score by the healthcare data processing module based on the processed health data.

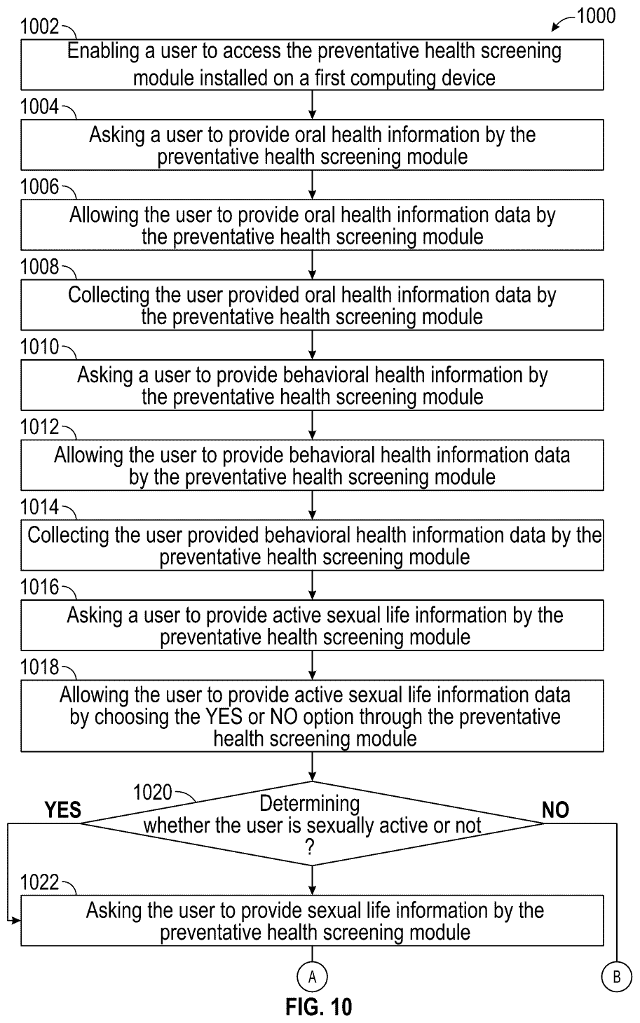

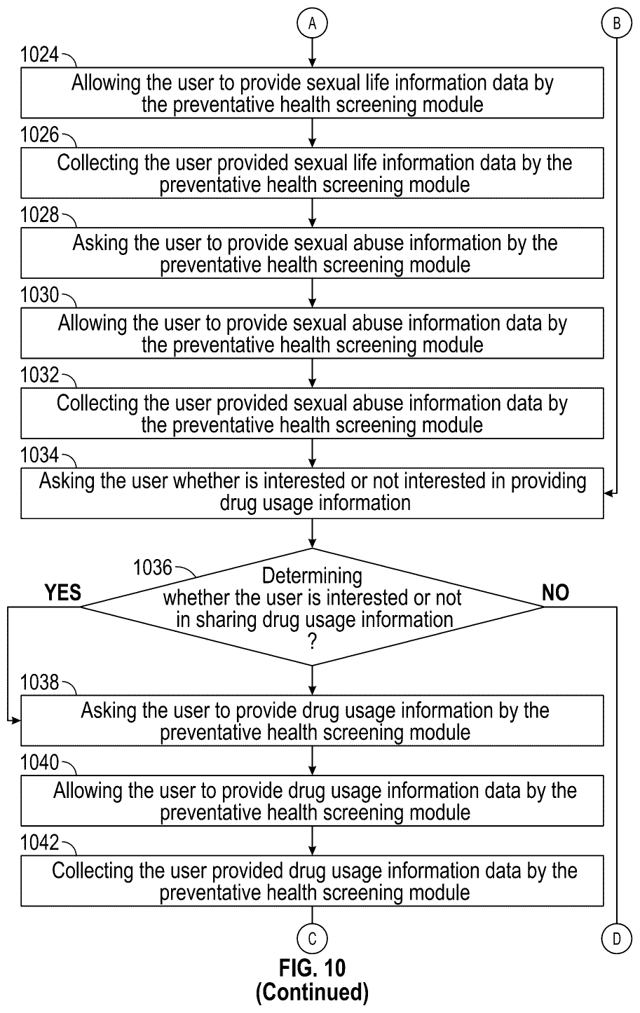

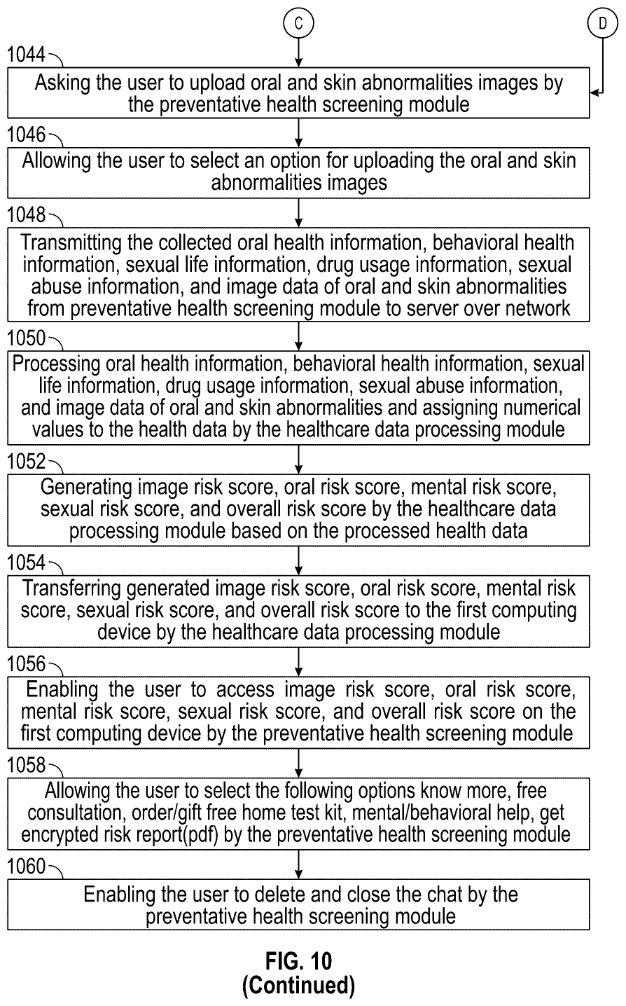

Referring to FIG. 10 is a flow diagram 1000 depicting a method for automated preventative health screenings, in accordance with one or more exemplary embodiments. The method 1000 may be carried out in the context of the details of FIG. 1, FIG. 2, FIG. 3, FIG. 4, FIG. 5A, FIG. 5B, FIG. 5C, FIG. 6A, FIG. 6B, FIG. 6C, FIG. 6D, FIG. 7, FIG. 8, and FIG. 9. However, the method 1000 may also be carried out in any desired environment. Further, the aforementioned definitions may equally apply to the description below. The exemplary method 1000 commences at step 1002, enabling a user to access the preventative health screening module installed on a first computing device. Thereafter at step 1004, asking a user to provide oral health information by the preventative health screening module. Thereafter at step 1006, allowing the user to provide oral health information data by the preventative health screening module. Thereafter at step 1008, collecting the user-provided oral health information data by the preventative health screening module. Thereafter at step 1010, asking a user to provide behavioral health information by the preventative health screening module. Thereafter at step 1012, allowing the user to provide behavioral health information data by the preventative health screening module. Thereafter at step 1014, collecting the user provided behavioral health information data by the preventative health screening module. Thereafter at step 1016, asking a user to provide active sexual life information by the preventative health screening module. Thereafter at step 1018, allowing the user to provide active sexual life information data by choosing the YES or NO option through the preventative health screening module. Determining whether the user is sexually active or not? at step 1020. If step 1020 is NO, the method goes to step 1034. If step 1020 is YES, then the method continues to step 1022, asking the user to provide sexual life information by the preventative health screening module. Thereafter at step 1024, allowing the user to provide sexual life information data by the preventative health screening module. Thereafter at step 1026, collecting the user provided sexual life information data by the preventative health screening module. Thereafter at step 1028, asking the user to provide sexual abuse information by the preventative health screening module. Thereafter at step 1030, allowing the user to provide sexual abuse information data by the preventative health screening module. Thereafter at step 1032, collecting the user provided sexual abuse information data by the preventative health screening module. Thereafter at step 1034, asking the user whether is interested or not interested in providing drug usage information. Determining whether the user is interested or not in sharing drug usage information? At step 1036. If step 1036 is NO, the method goes to step 1044. If step 1036 is YES, the method continues to step 1038. Thereafter at step 1038, asking the user to provide drug usage information by the preventative health screening module. Thereafter at step 1040, allowing the user to provide drug usage information data by the preventative health screening module. Thereafter at step 1042, collecting the user provided drug usage information data by the preventative health screening module. Thereafter at step 1044, asking the user to upload oral and skin abnormalities images by the preventative health screening module. Thereafter at step 1046, allowing the user to select an option for uploading the oral and skin abnormalities images. Thereafter at step 1048, transmitting the collected oral health information, behavioral health information, sexual life information, drug usage information, sexual abuse information, and image data of oral and skin abnormalities from the preventative health screening module to a server over a network. Thereafter at step 1050, processing oral health information, behavioral health information, sexual life information, drug usage information, sexual abuse information, and image data of oral and skin abnormalities and assigning numerical values to the health data by the healthcare data processing module. Thereafter at step 1052, generating Image risk score, Oral Risk Score, mental risk score, Sexual Risk Score, and Overall risk score by the healthcare data processing module based on the processed health data. Thereafter at step 1054, transferring generated Image risk score, Oral Risk Score, mental risk score, Sexual Risk Score, and Overall risk score to the first computing device by the healthcare data processing module. Thereafter at step 1056, enabling the user to access the Image risk score, Oral Risk Score, mental risk score, Sexual Risk Score, and Overall risk score on the first computing device through the preventative health screening module. Thereafter at step 1058, allowing the user to select the following options know more, free consultation, order/gift free home test kit (securely), mental/behavioral help, get encrypted risk report (pdf) by the preventative health screening module. Thereafter at step 1060, enabling the user to Delete and Close the chat by the preventative health screening module.

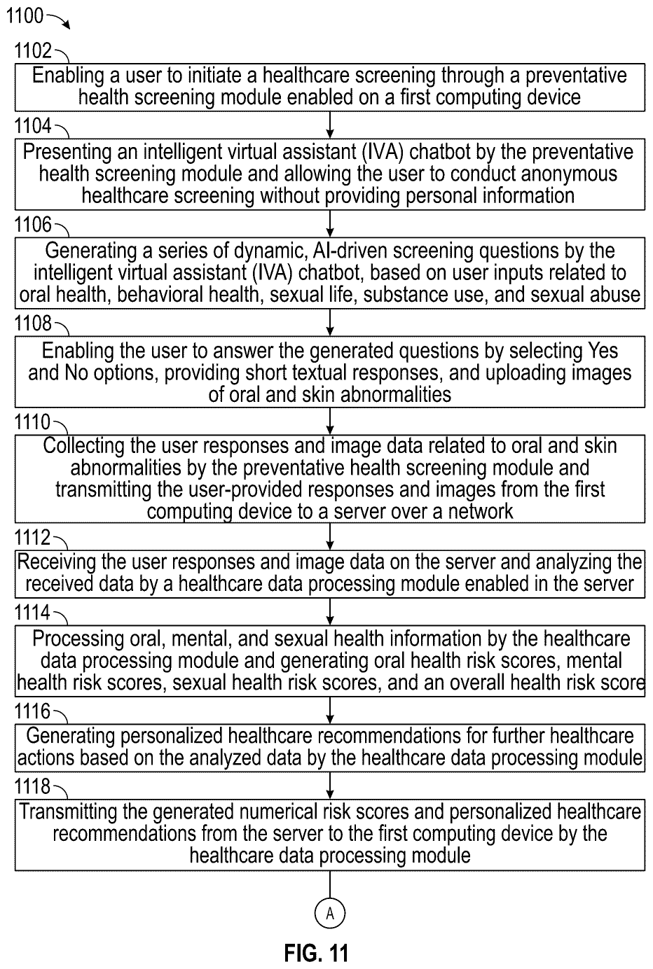

Referring to FIG. 11 is a flow diagram depicting a method for method for automated preventative health screening and data-driven health risk analysis, in accordance with one or more exemplary embodiments. The method 1100 may be carried out in the context of the details of FIG. 1, FIG. 2, FIG. 3, FIG. 4, FIG. 5A, FIG. 5B, FIG. 5C, FIG. 6A, FIG. 6B, FIG. 6C, FIG. 6D, FIG. 7, FIG. 8, FIG. 9 and FIG. 10. However, the method 1100 may also be carried out in any desired environment. Further, the aforementioned definitions may equally apply to the description below.

The exemplary method 1100 commences at step 1102, enabling the user to initiate anonymous screening process through a preventative health screening module enabled on a first computing device. Thereafter at step 1104, presenting an intelligent virtual assistant (IVA) chatbot by the preventative health screening module and allowing the user to conduct anonymous screening process without providing personal information. Thereafter at step 1106, generating a series of dynamic, AI-driven screening questions by the intelligent virtual assistant (IVA) chatbot, based on user inputs related to oral health, behavioral health, sexual life, substance use, and sexual abuse. Thereafter at step 1108, enabling the user to answer the generated questions by selecting Yes and No options, providing short textual responses, and uploading images of oral and skin abnormalities. Thereafter at step 1110, collecting the user responses and image data related to oral and skin abnormalities by the preventative health screening module and transmitting the user-provided responses and images from the first computing device to a server over a network. Thereafter at step 1112, receiving the user responses and image data on the server and analyzing the received data by a healthcare data processing module enabled in the server. Thereafter at step 1114, processing oral, mental, and sexual health information by the healthcare data processing module and generating oral health risk scores, mental health risk scores, sexual health risk scores, and an overall health risk score. Thereafter at step 1116, generating personalized healthcare recommendations for further healthcare actions based on the analyzed data by the healthcare data processing module. Thereafter at step 1118, transmitting the generated numerical risk scores and personalized healthcare recommendations from the server to the first computing device by the healthcare data processing module. Thereafter at step 1120, displaying the generated numerical risk scores and personalized healthcare recommendations to the user through the preventative health screening module on the first computing device. Thereafter at step 1122, enabling healthcare providers and case managers to access the user's health data, including the generated risk scores and personalized healthcare recommendations, based on the user's approval, through a second computing device and a third computing device. Thereafter at step 1124, allowing healthcare providers and case managers to manage and make informed decisions based on comprehensive screening results, using the preventative health screening module on the second computing device and the third computing device.

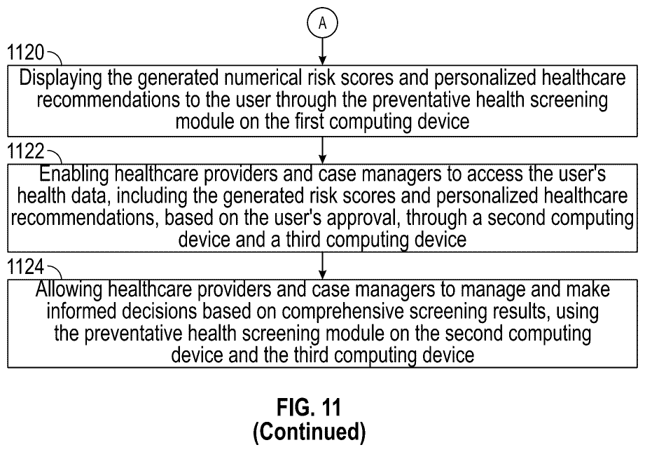

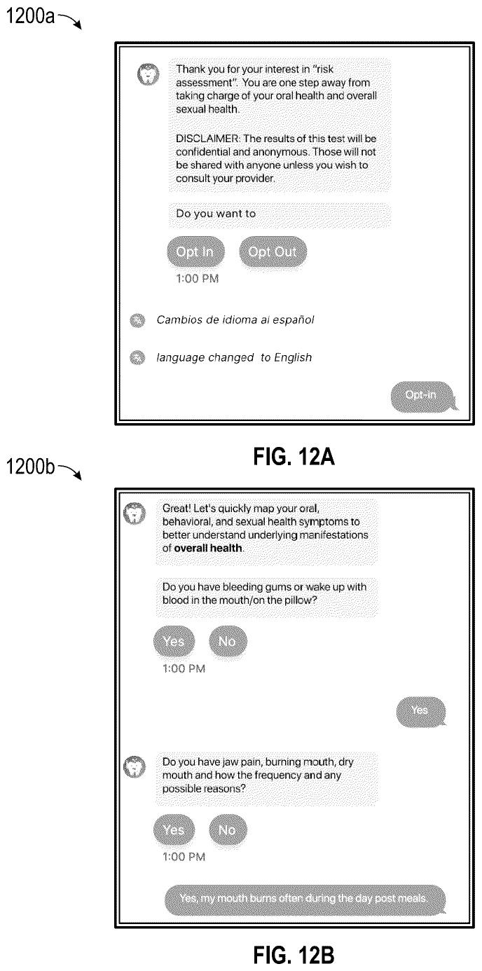

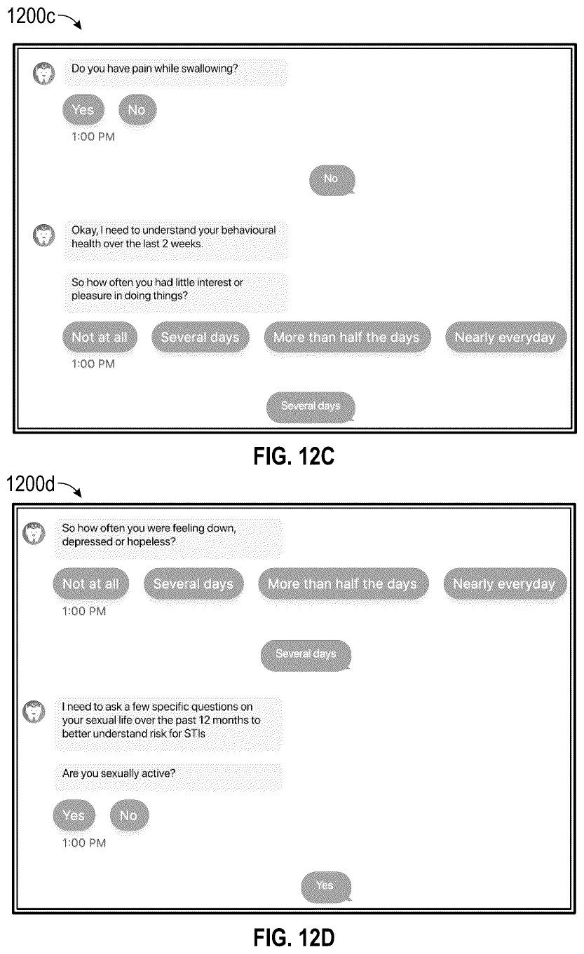

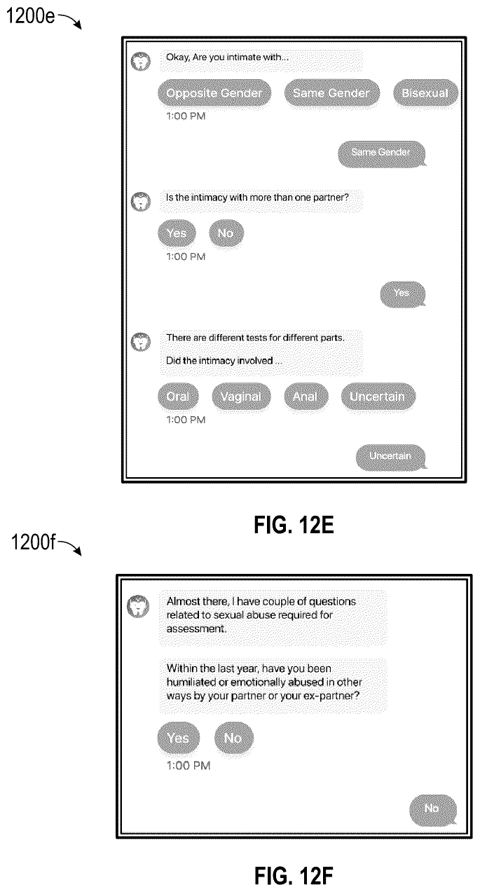

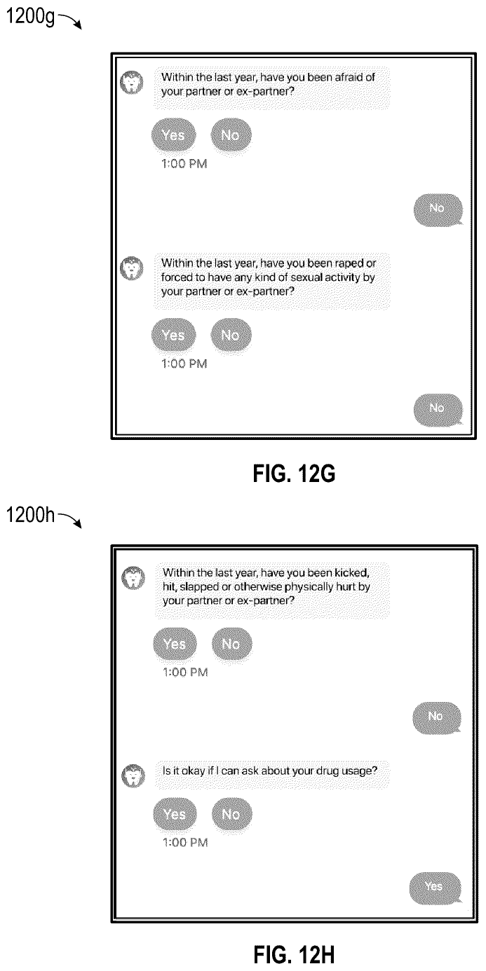

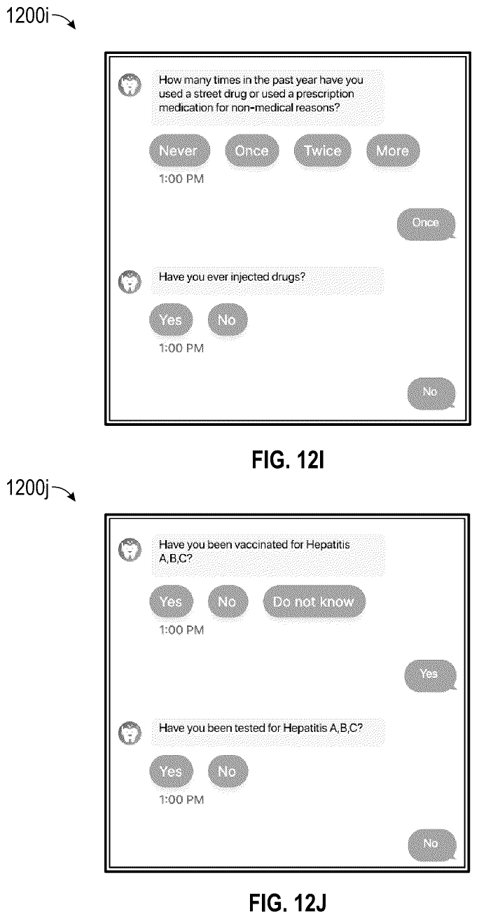

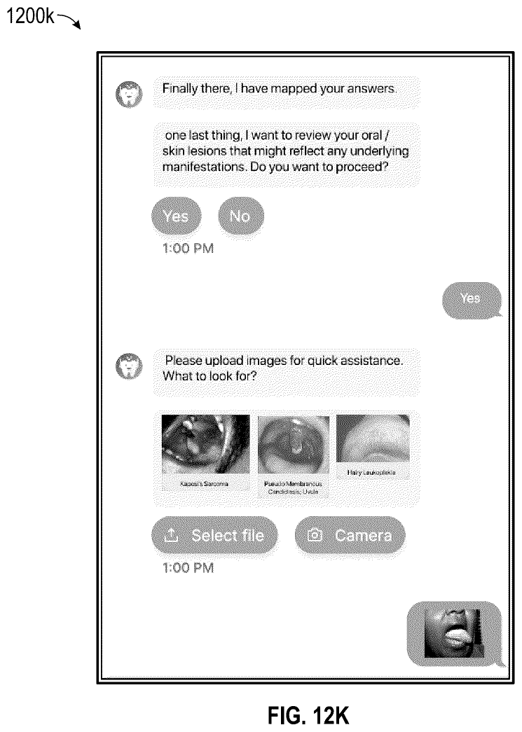

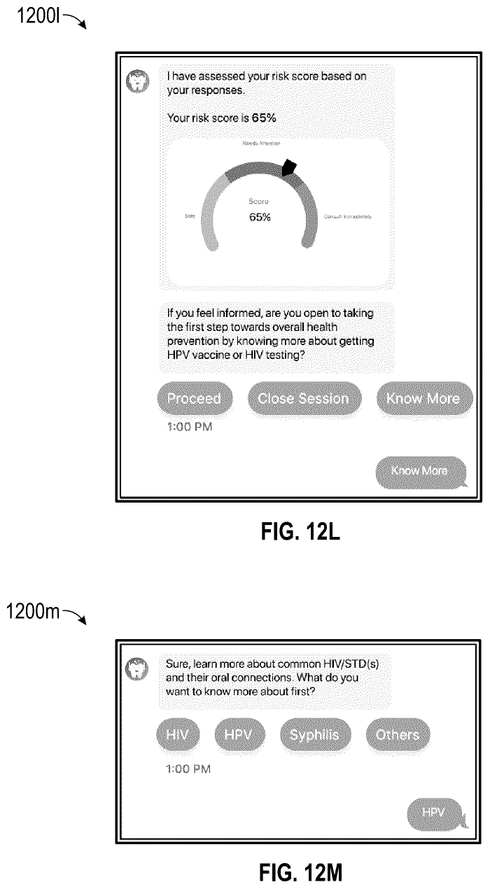

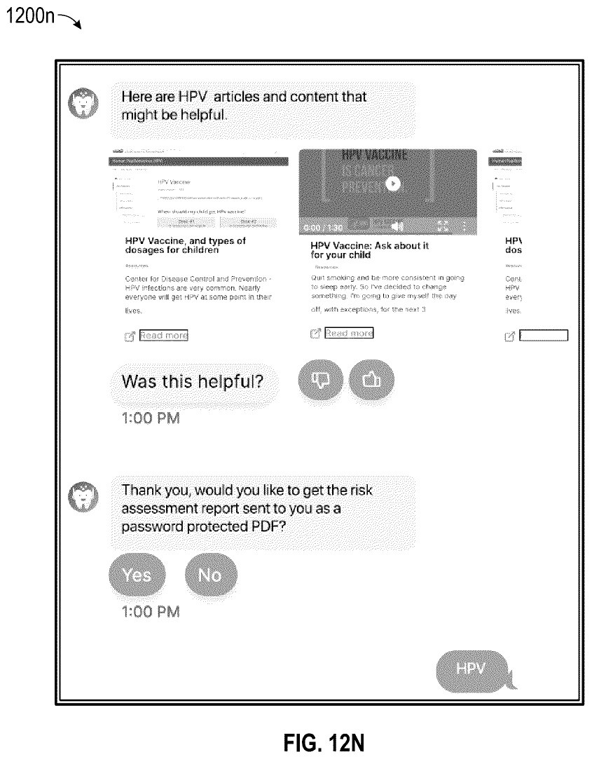

Referring to FIG. 12A to 12O are example screens depicting embodiments of the user interface module, in accordance with one or more exemplary embodiments. Enabling a user to access the preventative health screening module installed on a first computing device. asks a user to provide oral health information by the preventative health screening module. allowing the user to provide oral health information data by the preventative health screening module. collecting the user-provided oral health information data by the preventative health screening module. asking a user to provide behavioral health information by the preventative health screening module. allowing the user to provide behavioral health information data by the preventative health screening module. collecting the user-provided behavioral health information data by the preventative health screening module, asking a user to provide active sexual life information by the preventative health screening module. allowing the user to provide active sexual life information data by choosing the YES or NO option through the preventative health screening module. Determining whether the user is sexually active or not? at. If step is NO, the method goes to step. If step is YES, then the method continues to step, asking the user to provide sexual life information by the preventative health screening module. allowing the user to provide sexual life information data by the preventative health screening module. collecting the user provided sexual life information data by the preventative health screening module. Asking the user to provide sexual abuse information by the preventative health screening module, allowing the user to provide sexual abuse information data by the preventative health screening module, collecting the user provided sexual abuse information data by the preventative health screening module, asking the user whether is interested or not interested in providing drug usage information. enabling the user to access the Image risk score, Oral Risk Score, mental risk score, Sexual Risk Score, and Overall risk score on the first computing device through the preventative health screening module. allowing the user to select the following options know more, free consultation, order/gift free home test kit (securely), mental/behavioral help, and get an encrypted risk report (pdf) by the preventative health screening module. enabling the user to Delete and Close the chat through the preventative health screening module.

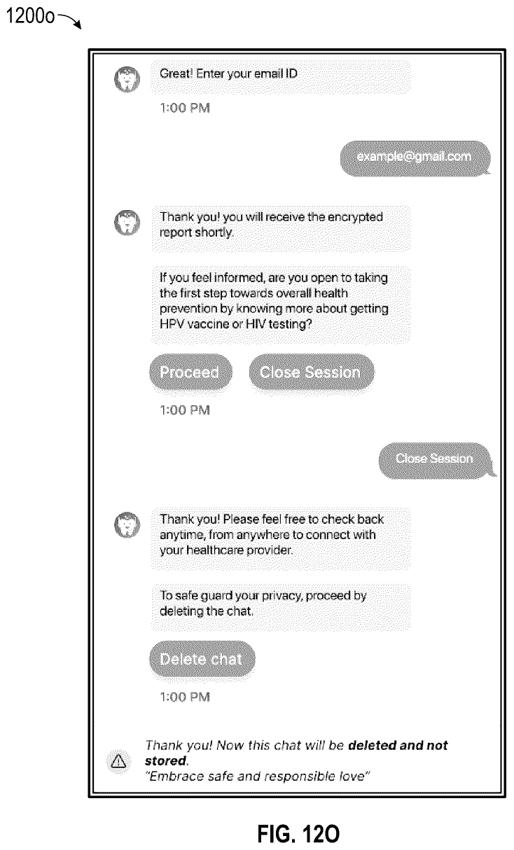

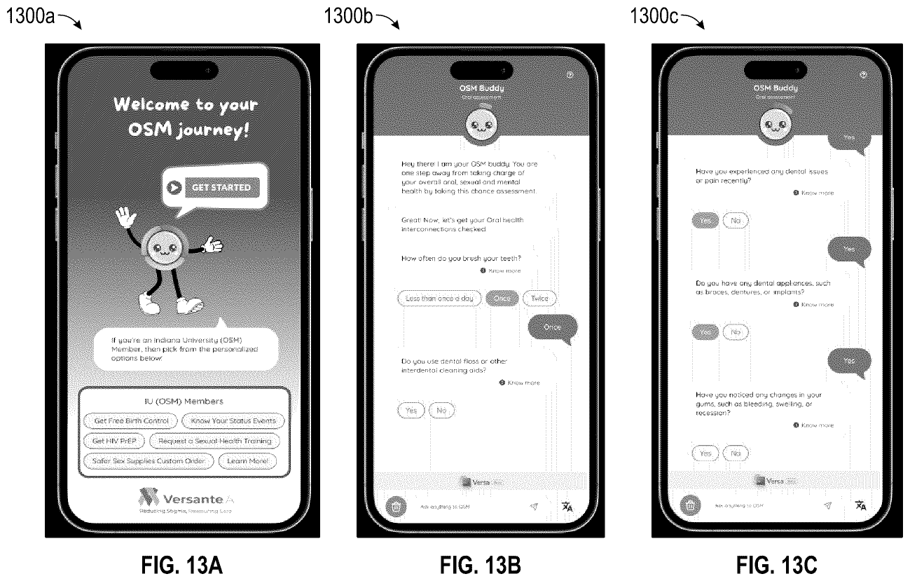

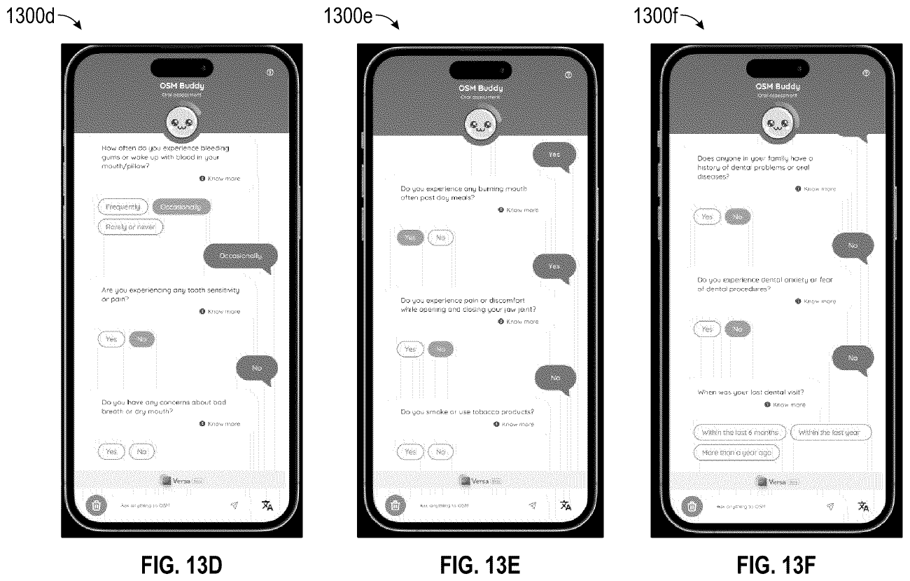

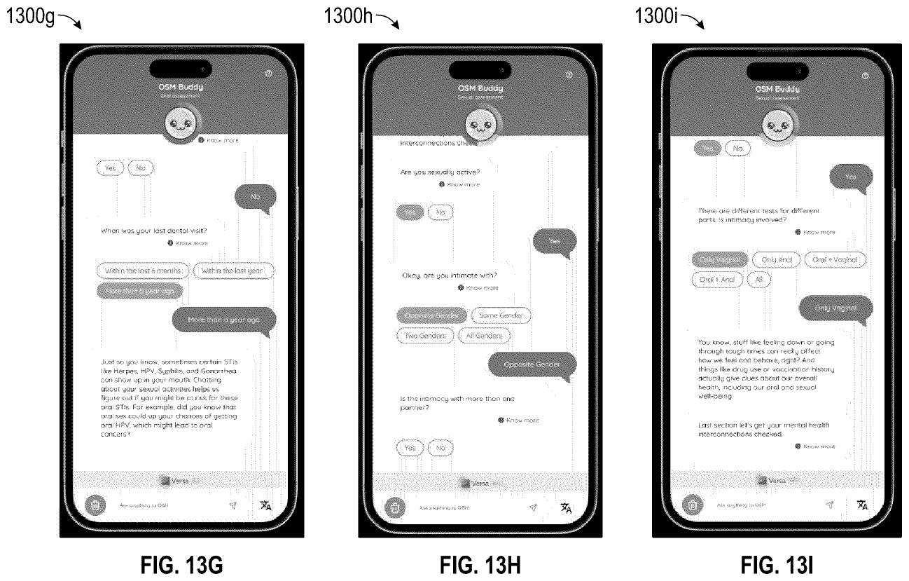

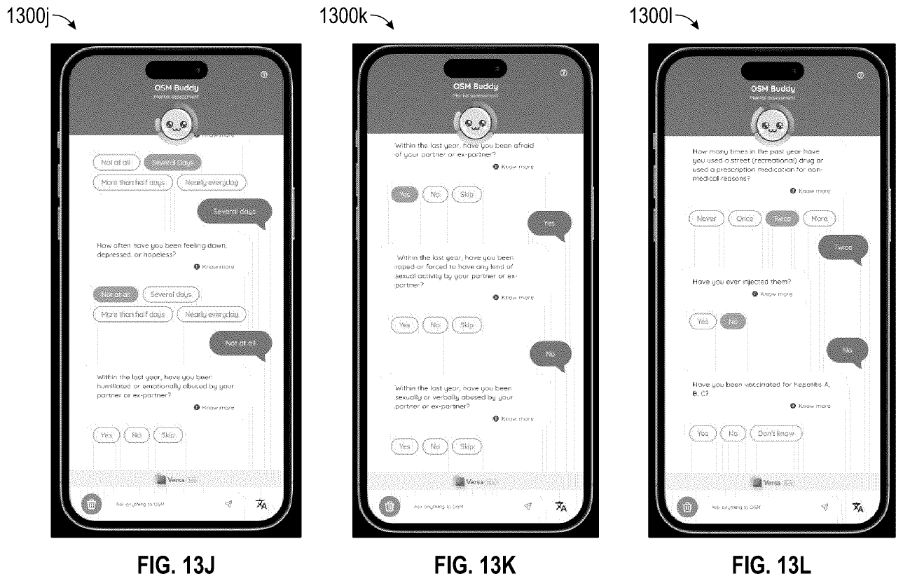

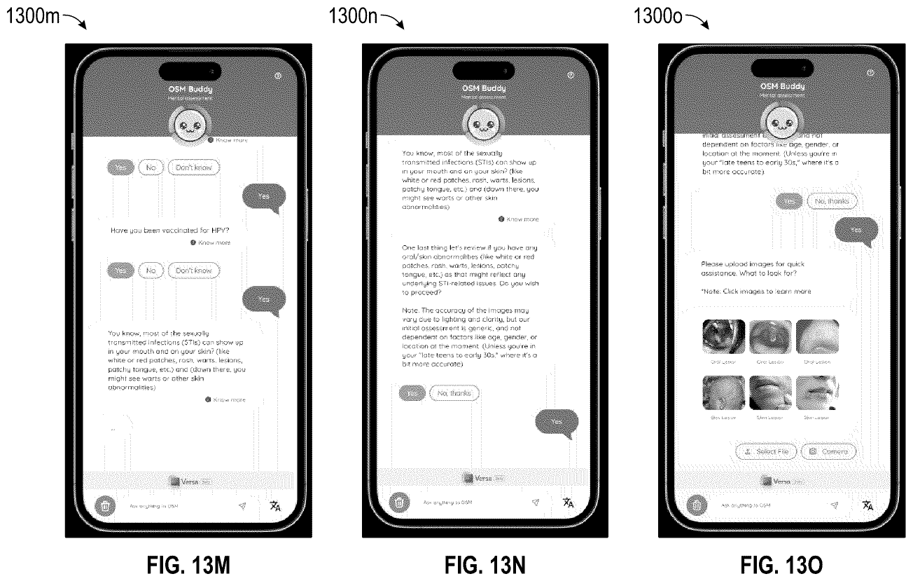

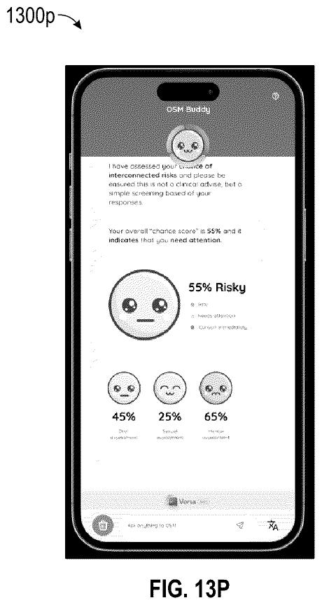

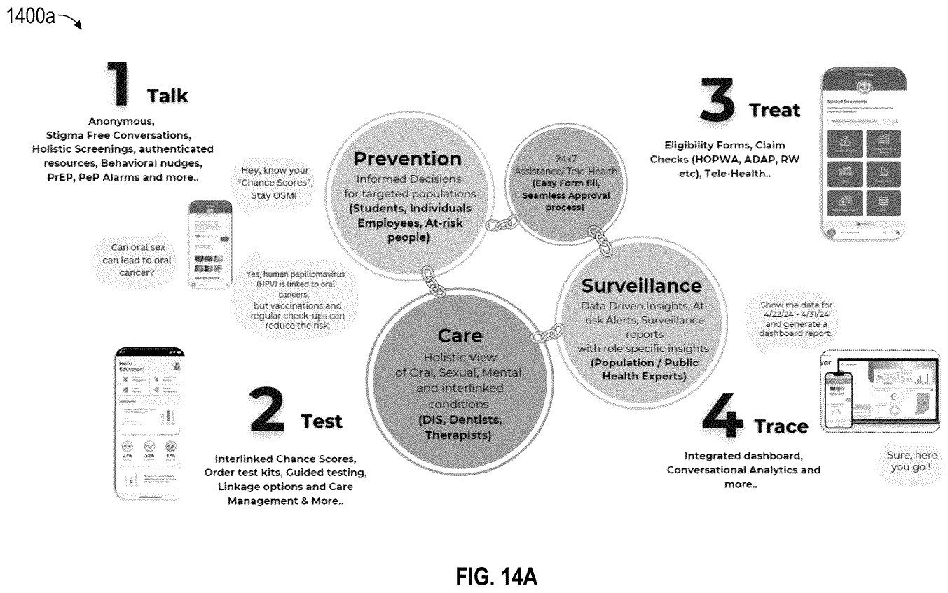

Referring to FIG. 13A to 13P are example screens 1300a to 1300p depicting embodiments of the user interface module that allow users to initiate and interact with various aspects of a preventative health screening, in accordance with one or more exemplary embodiments. FIG. 13A is an example screen depicting the initial user interface of the preventative health screening module 112. The screen welcomes users to their personalized OSM (Outreach, Screening, and Management) journey, inviting them to begin their anonymous screening process with a “Get Started” button prominently displayed. Below the welcome message, the interface includes personalized options for Indiana University OSM members, offering various healthcare services such as: Free Birth Control, Know Your Status Events, Getting HIV PrEP, requesting Sexual Health Training, Safer Sex Supplies Custom Order, Learn More! The design incorporates a friendly and approachable mascot, enhancing user engagement. This screen emphasizes accessibility and personalization, allowing users to quickly select from pre-configured health services without needing to sign in.

Referring to FIG. 13B is an example screen 1300b illustrating the interactive user interface of the preventative health screening module 112, specifically focused on oral health analysis. The screen features an introductory message from preventative health screening module 112, the virtual assistant, encouraging users to take control of their overall, oral, sexual, and mental health by participating in the anonymous screening process. The user is prompted with a health-related question: “How often do you brush your teeth?” The interface provides multiple-choice options in a user-friendly bubble format, including Less than once a day, once (selected by the user), or Twice and the interaction continues with a follow-up question: “Do you use dental floss or other interdental cleaning aids?” accompanied by the response options Yes and No, enabling users to easily provide feedback on their oral hygiene habits. A speech bubble format for easy interaction with preventative health screening module 112, guiding users through the oral health analysis step-by-step. A “Know more” option next to each question, allowing users to access additional information if needed before answering the questions. Navigation and action buttons at the bottom, such as a trash icon to reset the interaction, a language selection icon for real-time translation, and an input field for additional questions users may want to ask the preventative health screening module 112 directly.

Referring to FIG. 13C illustrates the interaction between the user and the preventative health screening module 112, focusing on the oral health analysis. The user is prompted to provide specific details related to their oral health status. This figure demonstrates how the chatbot collects relevant health information step-by-step through a series of conversational questions. The first question asks: “Have you experienced any dental issues or pain recently?” The user is given two options, Yes or No, with a tooltip option (via Know more) to provide further clarification on what constitutes dental issues or pain. The user selects Yes, indicating they have had recent dental issues. The second question follows up by asking: “Do you have any dental appliances, such as braces, dentures, or implants?” Again, the chatbot provides the Yes or No options, with an additional Know more feature to explain the types of dental appliances. The user selects Yes to confirm that they use such appliances. The third question seeks more specific information about the user's gum health: “Have you noticed any changes in your gums, such as bleeding, swelling, or recession?” The options remain consistent with previous questions, allowing the user to choose Yes or No, alongside the Know more option to clarify symptoms. At the bottom of the screen. The preventative health screening module 112 guiding the user through a conversational flow to make the interaction feel personalized and supportive. The option to Ask anything to preventative health screening module 112 via a text input box at the bottom, ensuring that users can ask additional questions beyond the standard prompts. Icons for language change and deletion/reset, enabling the user to customize the experience and control their data.

Referring to FIG. 13D illustrates a continuation of the oral health analysis conducted by preventative health screening module 112. In this figure, the preventative health screening module 112 gathers further details about the user's oral health, specifically focusing on issues related to gum health, tooth sensitivity, and concerns about bad breath or dry mouth. The first question asks: “How often do you experience bleeding gums or wake up with blood in your mouth/pillow?” The user is presented with three options: Frequently, occasionally (selected by the user), Rarely or never Additionally, the Know More option is available to explain common causes and symptoms of bleeding gums, helping users to better understand the condition before answering. The second question asks: “Are you experiencing any tooth sensitivity or pain?” The options provided are Yes or No, with the Know more option giving further information on what constitutes tooth sensitivity and its potential causes. In this instance, the user selects No, indicating they are not experiencing sensitivity or pain. The third question in this figure addresses concerns about bad breath or dry mouth: “Do you have any concerns about bad breath or dry mouth?” Similar to the previous questions, the user can respond with Yes or No, while the Know more option helps clarify possible underlying causes and related symptoms. The user selects No for this question as well. At the bottom of the screen, several key interface elements enhance user control and engagement: The Ask anything to input field allows users to type additional questions or seek clarification at any point during the anonymous screening process. The trash icon provides an option to reset or delete the current conversation, offering users full control over their interactions. The language change option enables users to switch to their preferred language in real-time, ensuring accessibility for a diverse audience. The conversational design of preventative health screening module 112 creates a smooth flow, encouraging users to provide honest and accurate information regarding their oral health. The use of familiar language and helpful prompts ensures that users feel comfortable sharing their health information, making the system more effective in providing personalized healthcare recommendations.

Referring to FIG. 13E illustrates an extension of the oral health analysis conducted by preventative health screening module 112, focusing on additional oral health symptoms and lifestyle habits that may influence the user's overall health. This figure shows a continuation of the conversational interaction between the user and the preventative health screening module 112. The first question asks: “Do you experience any burning mouth often post day meals?” The user is provided with the options Yes and No, and selects Yes to indicate that they experience burning sensations in their mouth. A Know more option is available, which provides the user with additional information on the possible causes of burning mouth, such as acidic foods, dehydration, or oral infections. The second question follows up by asking: “Do you experience pain or discomfort while opening and closing your jaw joint?” The user is again provided with the Yes and No options, and selects No for this question, indicating that they do not have issues with jaw pain or discomfort. The Know more feature provides further insights into conditions like temporomandibular joint (TMJ) disorder, which may cause jaw pain or discomfort. The third question addresses the user's lifestyle habits: “Do you smoke or use tobacco products?” The user is given Yes and No options, with the Know more option available to provide information on the health risks associated with smoking or tobacco use, such as increased risk of gum disease, tooth loss, and oral cancer. In this case, the user selects No, indicating they do not use tobacco products. The Ask anything input field at the bottom allows users to ask additional questions or seek clarification beyond the prompted questions, ensuring that the anonymous screening process remains interactive and user-driven. The trash icon allows users to delete or reset the chat at any time, reinforcing the platform's focus on user control and privacy. The language change icon enables the user to switch between languages, ensuring that the platform remains accessible to a diverse audience.

Referring to FIG. 13F illustrates an extended interaction between the user and preventative health screening module 112, the virtual healthcare assistant, as part of the oral health analysis. This figure focuses on gathering family history, dental anxiety, and dental visit data, all of which contribute to a comprehensive oral health profile for the user. The first question asks: “Does anyone in your family have a history of dental problems or oral diseases?” The user is given the options Yes or No, and in this case, they select No. The Know more option allows the user to access further information about common hereditary dental conditions and how family history can influence oral health. This feature helps users make more informed decisions while responding. The second question addresses dental anxiety: “Do you experience dental anxiety or fear of dental procedures?” The user again has the choice between Yes or No, and they select No. The Know more option provides additional context regarding dental anxiety, how it manifests, and potential coping strategies for those who experience it. This information helps users feel reassured and better understand their situation. The third question focuses on the timing of the user's last dental visit: “When was your last dental visit?” The options provided are: Within the last 6 months, within the last year, more than a year ago. The user can choose the response that best reflects their most recent dental appointment. This information helps the healthcare system gauge whether the user is keeping up with routine check-ups or might need additional encouragement to schedule a visit. The Know more option here could provide insights into the importance of regular dental check-ups and how they contribute to preventing oral health issues.

Referring to FIG. 13G continues the oral health analysis conducted by preventative health screening module 112 and incorporates educational messaging about the risks associated with sexually transmitted infections (STIs) that may affect oral health. This figure focuses on the user's last dental visit and provides contextual information on oral health risks related to sexual activity. The first interaction on this screen asks the user to confirm when their last dental visit occurred. The user is provided with three options: Within the last 6 months, Within the last year, or More than a year ago (selected by the user), The Know More option is available to educate the user about the importance of regular dental visits and their role in maintaining overall oral health. Once the user selects More than a year ago, the chatbot continues with an important educational message about oral health risks related to STIs. The message states: “Just so you know, sometimes certain STIs like Herpes, HPV, Syphilis, and Gonorrhea can show up in your mouth. Chatting about your sexual activities helps us figure out if you might be at risk for these oral STIs. For example, did you know that oral sex could up your chances of getting oral HPV, which might lead to oral cancers?” This message serves to inform the user about the potential oral health consequences of STIs, linking sexual health to oral health risks. By presenting this information conversationally, preventative health screening module 112 encourages users to consider their sexual activity and its potential impact on oral health, prompting them to provide relevant sexual health information in subsequent steps.

Referring to FIG. 13H depicts the interaction between the user and preventative health screening module 112 during the sexual health analysis. This figure demonstrates how the chatbot collects key information regarding the user's sexual activity, preferences, and number of partners. This data helps to evaluate the user's risk of sexually transmitted infections (STIs) and other sexual health concerns. The first question asks: “Are you sexually active?” The user is given the options Yes and No, and in this case, they select Yes. A Know more option is available to provide users with additional context on what constitutes sexual activity and why this information is important for health analysis. After confirming sexual activity, the chatbot asks: “Okay, are you intimate with?” This question seeks to gather information about the user's sexual orientation and preference in partners. The options provided include: Opposite gender (selected by the user), same gender, two genders, all genders. The Know more option allows the user to gain a better understanding of how gender preferences and sexual orientation can relate to their health and risk factors. By offering a range of gender options, the system ensures inclusivity and caters to a wide spectrum of users. The next question follows up by asking: “Is the intimacy with more than one partner?” The user can choose Yes or No based on their personal situation. The Know more option provides additional information regarding the potential risks of having multiple partners, such as the increased likelihood of contracting STIs, and the importance of regular testing and safe sexual practices.

Referring to FIG. 13I showcases a continuation of the sexual health analysis conducted by preventative health screening module 112, focusing on the nature of the user's sexual intimacy and the impact of overall well-being on their health. The figure illustrates how the system collects vital information related to sexual health and introduces the next section of the anonymous screening process, which covers mental health. The first interaction in this figure asks: “There are different tests for different parts. Is intimacy involved?” The user is given the options Yes or No, with the user selecting Yes. A Know more option provides further context on why the system is asking about different forms of intimacy and how it relates to specific health tests. After confirming intimacy, the chatbot prompts the user to clarify the type of intimacy by asking: “Which type?” The user is presented with multiple options to describe their form of sexual activity: Only Vaginal (selected by the user), Only Anal, Oral+Vaginal, Oral+Anal, All. These options allow the system to tailor recommendations and testing suggestions based on the specific sexual practices of the user. For instance, the risk of certain sexually transmitted infections (STIs) may differ depending on the nature of sexual activity, and specific tests may be recommended for each type of intimacy. The Know more option gives users additional information about the importance of tailored testing for different types of sexual activity. After collecting the intimacy data, preventative health screening module 112 introduces a broader health perspective: “You know, stuff like feeling down or going through tough times can really affect how we feel and behave, right? And things like drug use or vaccination history actually give clues about our overall health, including our oral and sexual well-being.” This message ties together the user's mental, behavioral, and sexual health, emphasizing the interconnected nature of these aspects in determining overall well-being. Finally, the preventative health screening module 112 transitions the user to the mental health analysis section by stating: “Last section, let's get your mental health interconnections checked.” This smooth transition ensures that the user understands the importance of addressing mental health, following the sexual health analysis. The Know more option can provide the user with additional context on how mental health factors, such as stress, anxiety, or depression, may impact their sexual and overall health.

Referring to FIG. 13J illustrates a continuation of the mental health and emotional well-being analysis conducted by preventative health screening module 112. The focus here is on identifying depressive symptoms and emotional abuse, which are critical components in assessing a user's overall mental health. The first question asks: “How often have you been feeling down, depressed, or hopeless?” The user is given the following options: Not at all, several days (selected by the user), More than half days, Nearly every day. The Know more option is available for the user to access additional information on what it means to feel down, depressed, or hopeless. This helps users accurately identify and report any mental health issues they may be experiencing. This question aims to screen for depression and other mood disorders by gauging the frequency of these negative feelings. The second question asks: “How often have you been feeling down, depressed, or hopeless?” The user selects Not at all, indicating that they do not often experience such feelings. The Know more option remains available to explain the potential causes and symptoms of depression, offering additional support and context. The third question shifts focus to potential emotional abuse: “Within the last year, have you been humiliated or emotionally abused by your partner or ex-partner?” The user is given three response options: Yes, No, Skip. The Skip option is an important feature, allowing users to bypass this question if they feel uncomfortable answering it. The Know more option explains what constitutes emotional abuse and the potential psychological effects, helping users understand this sensitive issue and encouraging them to seek help if necessary.

Referring to FIG. 13K continues the mental health analysis conducted by preventative health screening module 112, specifically focusing on identifying abusive relationships and trauma-related experiences. This figure presents a series of sensitive questions that help assess whether the user has experienced emotional, sexual, or verbal abuse within the past year. The first question asks: “Within the last year, have you been afraid of your partner or ex-partner?” The user is provided with three response options: Yes, No, Skip. In this case, the user selects Yes to indicate that they have felt fear in their relationship. The Skip option allows users to bypass this sensitive question if they are uncomfortable answering it, while the Know more option provides context about the psychological effects of fear in relationships and why this question is important for the analysis. The second question follows up with: “Within the last year, have you been raped or forced to have any kind of sexual activity by your partner or ex-partner?” The response options remain the same Yes, No, or Skip and in this case, the user selects No. The Know more option explains the definitions of sexual coercion and rape, helping users recognize abusive behaviors that may not be immediately apparent or easy to discuss. The third question asks: “Within the last year, have you been sexually or verbally abused by your partner or ex-partner?” Again, the user is provided with Yes, No, or Skip options, and the user selects No. The Know more option here provides additional information on how sexual or verbal abuse can manifest in relationships, encouraging users to reflect on their experiences and seek help if needed.

Referring to FIG. 13L illustrates the continuation of the mental health analysis, focusing on the user's drug use history and vaccination status. The preventative health screening module 112 guides the user through these questions in a supportive manner, gathering essential health data related to substance use and vaccinations, which are critical to assessing the user's overall health. The first question asks: “How many times in the past year have you used a street (recreational) drug or used a prescription medication for non-medical reasons?” The user is provided with four response options: Never, Once, twice (selected by the user), More. The Know more option is available to provide users with context on what constitutes recreational drug use or misuse of prescription medications. By gathering this information, the system assesses the user's risk for substance-related health issues. The conversational tone and multiple-choice format make it easier for users to disclose sensitive information comfortably. The second question follows up by asking: “Have you ever injected them?” This question is crucial for understanding the user's risk of contracting bloodborne diseases like HIV or Hepatitis C, which are commonly associated with injected drug use. The user is given Yes and No options and selects No. The Know more option provides further explanation about the risks associated with intravenous drug use and why this question is significant. The third question shifts focus to the user's vaccination status, asking: “Have you been vaccinated for hepatitis A, B, C?” The response options provided are: Yes, no, don't know. The user can select the appropriate option based on their vaccination history, with Don't know available for users who are unsure of their vaccination status. The Know more option provides educational information about Hepatitis A, B, and C, explaining the importance of vaccination in preventing these potentially life-threatening diseases, especially for those with risk factors related to substance use or sexual health.

Referring to FIG. 13M illustrates the continuation of the mental health and vaccination analysis conducted by preventative health screening module 112, focusing on human papillomavirus (HPV) vaccination and sexually transmitted infection (STI) awareness. The chatbot guides the user through the anonymous screening process, gathering essential vaccination information and educating the user about visible symptoms of STIs. The first question asks: “Have you been vaccinated for HPV?” (Human Papillomavirus). The user is presented with three response options: Yes (selected by the user), No, Don't know. The Know more option provides additional context, helping the user understand the importance of the HPV vaccine in preventing certain types of cancer, including cervical, anal, and oropharyngeal cancers, as well as genital warts. This vaccination question is critical because HPV is one of the most common STIs, and early vaccination is a key preventive measure. After the user confirms they have been vaccinated for HPV, the preventative health screening module 112 provides educational information, stating: “You know, most of the sexually transmitted infections (STIs) can show up in your mouth and on your skin? (like white or red patches, rash, warts, lesions, patchy tongue, etc.) and (down there, you might see warts or other skin abnormalities).” This message serves as an educational prompt to increase the user's awareness of STI symptoms that might not be immediately recognizable. It encourages users to monitor their oral and skin health for signs of possible infections and provides them with useful information on what to look for, including symptoms such as patches, lesions, warts, and rashes, which are common indicators of STIs like HPV, herpes, and syphilis.

Referring to FIG. 13N continues the sexual health and STI (sexually transmitted infection) awareness, where preventative health screening module 112 provides users with critical information about the symptoms of STIs and proceeds to offer an opportunity for the user to conduct a self-anonymous screening process for visible oral or skin abnormalities that might indicate an STI. The first message from preventative health screening module 112 reiterates the symptoms of STIs, stating: “You know, most of the sexually transmitted infections (STIs) can show up in your mouth and on your skin? (like white or red patches, rash, warts, lesions, patchy tongue, etc.) and (down there, you might see warts or other skin abnormalities).” This educational prompt reinforces earlier guidance, ensuring that the user is aware of common visual symptoms of STIs. The Know more option is available, offering the user the opportunity to access further information on how and why STIs manifest on the skin and oral regions. This prepares the user for the next step, which involves examining their own skin or oral health. The second message offers the user the chance to proceed with a more in-depth self-anonymous screening process: “One last thing: let's review if you have any oral/skin abnormalities (like white or red patches, rash, warts, lesions, patchy tongue, etc.) as that might reflect any underlying STI-related issues. Do you wish to proceed?” This invitation asks the user whether they would like to upload images of any visible abnormalities for further analysis. The message includes a Note, which states: “The accuracy of the images may vary due to lighting and clarity, but our initial analysis is generic, and not dependent on factors like age, gender, or location at the moment. (Unless you're in your ‘late teens to early 30s,’ where it's a bit more accurate).” This note helps manage user expectations, explaining that while the image-based analysis provides helpful insights, it is not definitive, particularly due to the variance in image quality. The user is provided with two response options: Yes (selected by the user), indicating that they wish to proceed with the self-anonymous screening process. No, thanks, giving the user the option to skip this step if they are uncomfortable or do not have any abnormalities to report. This step encourages users to take an active role in monitoring their own health, particularly with respect to sexually transmitted infections, while also respecting their privacy and comfort levels.

Referring to FIG. 13O continues the self-anonymous screening process initiated in the previous steps, where preventative health screening module 112 requests the user to upload images of any visible oral or skin lesions for quick assistance. This figure demonstrates how the system facilitates image-based analysis by providing visual examples of common lesions and offering tools to upload or capture images for review. The first prompt from preventative health screening module 112 asks the user to upload images: “Please upload images for quick assistance. What to look for?” This question invites the user to participate in the anonymous screening process by uploading pictures of any oral or skin abnormalities they may have noticed. Below this prompt, the preventative health screening module 112 provides visual examples of common lesions to help the user identify possible symptoms. The examples include: Oral lesions, such as white or red patches, warts, or other abnormalities visible in the mouth. Skin lesions, such as rashes, warts, or sores visible on different parts of the body. Each visual example has a label and is clickable, allowing users to access more information about what to look for. The note: “Click images to learn more” informs the user that they can click on any of the examples to gain further insight into what constitutes a potential STI-related lesion. Two options for uploading or capturing images are available: Select File: This button allows users to upload an existing image from their device for review. Camera: This button enables users to capture a new image using their device's camera, ensuring that they can provide real-time, up-to-date photos for the anonymous screening process.

Referring to FIG. 13P showcases the final results of the health screening conducted by the Preventative health screening module, where the system evaluates the user's responses and presents an overall “chance score” for potential health risks. This figure illustrates the feedback and guidance provided to the user based on their participation in the oral, sexual, and mental health analysis. The first message from the preventative health screening module states: “I have assessed your chance of interconnected risks and please be ensured this is not a clinical advice, but a simple screening based on your responses.” This clarifies that the analysis results are a preliminary screening and not a substitute for professional medical advice. The system reminds the user that the outcome is based on their inputs and encourages them to seek further consultation if necessary. The next message provides the user's overall “chance score,” which is displayed as a percentage. In this case, the system reports: “Your overall ‘chance score’ is 55% and it indicates that you need attention.” This overall score helps the user understand the combined risk across different health categories, signaling whether they need to take action based on the results. Below the overall score, the user is presented with a visual indicator showing a “55% Risky” analysis. The indicator includes three color-coded statuses: Green for “Safe,” meaning the user is likely at low risk. Yellow for “Needs attention,” meaning the user should monitor their health and consider seeking advice or care. Red for “Consult immediately,” meaning the user is at high risk and should seek immediate medical help. In this case, the user falls into the yellow category, which indicates that their health needs attention based on the analysis. The next section of the figure breaks down the user's individual scores for the three areas of analysis: Oral analysis: 45%, Sexual analysis: 25%, Mental analysis: 65%. Each score is represented by a corresponding icon with facial expressions that reflect the level of risk: The Oral analysis icon with a neutral face reflects a moderate risk score of 45%, indicating some concerns that should be addressed. The Sexual analysis icon with a smiling face reflects a lower risk score of 25%, suggesting that the user's sexual health appears to be in good condition. The Mental analysis icon with a concerned face reflects a higher risk score of 65%, indicating that mental health requires significant attention.

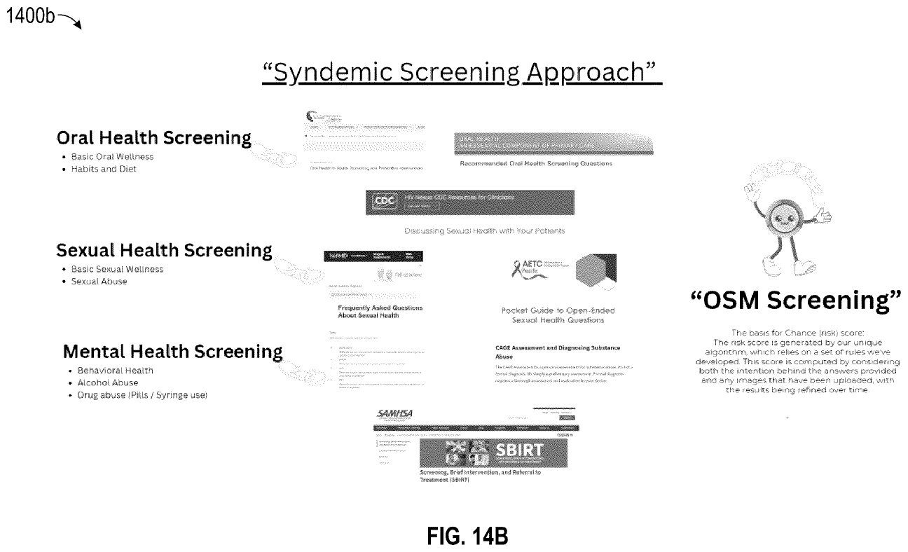

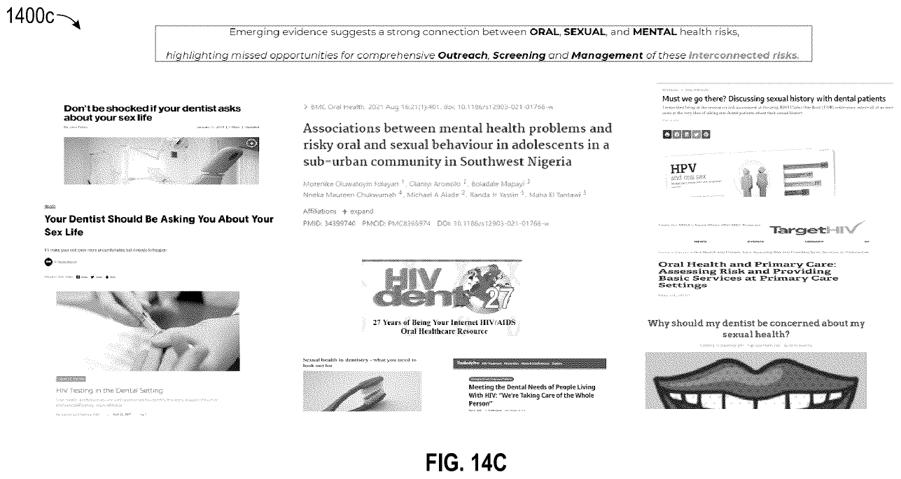

Referring to FIG. 14A is a schematic diagram illustrating a holistic model of an artificial intelligence-based system for automated preventative health screening and data-driven health risk analysis, in accordance with one or more exemplary embodiments. The holistic healthcare model encompasses four key stages: Talk, Test, Treat, and Trace. Each stage is interconnected to provide comprehensive healthcare support through conversations, screenings, treatments, and surveillance. The diagram visually depicts how users engage with the system, receive analysis results, and are guided toward appropriate healthcare resources, including prevention, care, and data-driven surveillance. It highlights the importance of informed decision-making, preventive measures, and seamless navigation through the healthcare system. The figure divides the process into four core stages: Talk, Test, Treat, and Trace, each supported by key features and actions aimed at enhancing user engagement, improving health outcomes, and enabling easy navigation through the healthcare system. Stage 1: Talk. The first section, titled “Talk,” emphasizes the anonymous nature of the platform, where users can engage in stigma-free conversations that address a variety of health topics, including oral, mental, and sexual health. It allows users to access holistic screenings and behavioral nudges through casual dialogues, helping them stay informed about key healthcare concerns such as HIV prevention (PrEP, PeP) and other behavioral health needs. This stage also introduces OSM Buddy's conversation features, where users can ask health-related questions. For example, a question such as “Can oral sex lead to oral cancer?” is answered with a clear and informative response: “Yes, human papillomavirus (HPV) is linked to oral cancers, but vaccinations and regular check-ups can reduce the risk.” The system engages the user with real-time nudges, promoting informed health decisions and reducing stigma around sensitive health topics. Stage 2: Test, the second section, titled “Test,” provides users with interlinked health scores through comprehensive screenings that evaluate various health aspects such as oral, sexual, and mental health. These interlinked chance scores provide a holistic view of the user's health risks. The system also offers the option to order test kits and participate in guided testing through seamless integration with the platform. In this stage, guided testing and the ability to order test kits make health screenings more accessible, allowing users to track and manage their health status remotely. Additionally, there is a reference to care management that helps guide users toward appropriate healthcare options based on their risk scores. Stage 3: Treat, the third section, “Treat,” focuses on providing users with access to telehealth services and support for eligibility forms, claim checks, and billing guidance. The system supports various claim processes such as HOPWA, ADAP, and RW, ensuring that users receive the financial assistance they need for treatment. Users are also guided through billing and coding processes (ICD, CPT, etc.) with the assistance of the system, which simplifies the administrative side of accessing healthcare services. The figure shows an example of the document upload interface, where users can submit relevant documentation to streamline their access to care and support. Stage 4: Trace, the final stage, “Trace,” involves the surveillance and monitoring of the user's health data. The system provides integrated dashboards and conversational analytics to healthcare providers, allowing for data-driven decision-making and ongoing monitoring of the user's health. Data-driven insights, such as alerts and surveillance reports, are shared with public health experts, providing them with the necessary tools to offer targeted healthcare interventions to populations at risk. The figure shows the user interacting with preventative health screening module 112, requesting a dashboard report for specific dates, which the system generates in real-time to provide actionable insights. This promotes efficient case management and enhances the overall care experience. The diagram includes a focus on prevention and care through informed decisions for targeted populations (students, at-risk individuals, employees, etc.). It offers a holistic view of oral, sexual, and mental health, encouraging users to take proactive steps toward maintaining overall well-being. The system provides 24×7 support through easy form filling and a seamless billing process, making it easier for users to access healthcare services at any time. Icons and text bubbles guide the user through their interactions with the system, reinforcing key points such as prevention, testing, treatment, and tracing, all of which contribute to a user-friendly and intuitive healthcare experience.

Referring to FIG. 14B is an example diagram 1400b illustrating the syndemic screening approach employed by the preventative health screening module, in accordance with one or more exemplary embodiments. The figure breaks down the health screening process into three core areas: Oral Health Screening, Sexual Health Screening, and Mental Health Screening, emphasizing their interconnectedness in overall health analysis. Additionally, the figure highlights the Screening process, where a custom algorithm generates a chance (risk) score based on user responses and uploaded images. This approach focuses on providing a comprehensive view of the user's health, taking into account multiple dimensions of wellness. This approach integrates three key health screening categories—oral, sexual, and mental health—to provide a holistic view of the user's overall well-being. The term “syndemic” highlights the interconnectedness of these health domains, reflecting how issues in one area may impact or exacerbate problems in another. The figure further explains how the OSM Screening process employs a custom algorithm to generate a chance (risk) score that reflects the user's potential health risks. Oral Health Screening, the first section of the figure outlines the Oral Health Screening process, which focuses on: Basic Oral Wellness: This includes general oral hygiene, dental care habits, and analysis of any symptoms related to oral health, such as lesions or gum issues. Habits and Diet: The system evaluates lifestyle factors such as the user's diet, consumption of sugary foods, and habits like smoking or alcohol consumption, which can influence oral health outcomes. The figure includes visual references to oral health screening tools, such as recommended oral health screening questions, to support evidence-based analysis. Sexual Health Screening, the second section covers Sexual Health Screening, which addresses: Basic Sexual Wellness: This includes questions related to the user's sexual practices, safe sex habits, and general sexual health awareness. Sexual Abuse: The system screens for any history of sexual abuse or coercion, helping identify trauma or risks that may impact both mental and physical health. The figure references resources from authoritative organizations like the CDC and sexual health guides, ensuring that the screening process aligns with current best practices and guidelines for assessing sexual health. Mental Health Screening, the third section explains mental health screening, which covers: Behavioral Health: The system assesses the user's emotional and psychological well-being, including signs of anxiety, depression, or other behavioral health issues. Alcohol Abuse: The screening includes questions about alcohol use, aiming to identify risky drinking patterns or dependencies. Drug Abuse (Pills/Syringe use): The system also asks users about drug use, including both prescription medication misuse and recreational drug use. The figure references the CAGE analysis for substance abuse, as well as resources like SBIRT (Screening, Brief Intervention, and Referral to Treatment) for further analysis and intervention. This multi-dimensional approach ensures that users receive a thorough health screening, addressing key areas of physical, sexual, and mental health that are often interconnected. The OSM Screening process is described. This part of the figure explains how the chance (risk) score is generated using a unique algorithm developed by the system. The algorithm takes into account: The intentions behind the answers provided by the user during the screening process.

Referring to FIG. 14C is an example diagram 1400c depicting the strong connection between oral, sexual, and mental health risks, in accordance with one or more exemplary embodiments. The figure emphasizes how these health domains are interlinked and the missed opportunities for comprehensive Outreach, Screening, and Management of these interconnected risks. It references various studies, articles, and expert opinions that call for a more integrated approach to health care, particularly addressing how oral health professionals should engage in discussions about sexual health and mental health with patients. This visual emphasizes the importance of holistic health analysis to improve outcomes. The statement: “Emerging evidence suggests a strong connection between ORAL, SEXUAL, and MENTAL health risks, highlighting missed opportunities for comprehensive Outreach, Screening, and Management of these interconnected risks.” This introduces the central theme of the figure, which is the urgent need to address these interrelated health risks in an integrated manner, rather than treating them as isolated concerns. Oral, Sexual, and Mental Health Articles and Studies. Oral Health and Sexual Health: One of the featured articles asks, “Don't be shocked if your dentist asks about your sex life,” emphasizing the role oral health professionals should play in screening for sexual health issues. This article suggests that, despite the discomfort it may cause, it is important for dentists to address topics such as sexual health because of the oral manifestations of certain sexually transmitted infections (STIs), including HPV. Another article titled “Your Dentist Should Be Asking You About Your Sex Life” reiterates the idea that oral health professionals are well-positioned to identify potential risks associated with sexual health, particularly in light of the oral health impacts of STIs like HPV and HIV. Interconnected Health Risks and Research: A research article titled “Associations between mental health problems and risky oral and sexual behaviour in adolescents in a sub-urban community in Southwest Nigeria” delves into the links between mental health challenges and risky sexual and oral behaviors. This study provides empirical evidence that supports the need for an integrated approach to addressing mental, sexual, and oral health, especially in at-risk populations like adolescents. A piece on the Target HIV platform examines “Oral Health and Primary Care: Assessing Risk and Providing Basic Services at Primary Care Settings,” suggesting that primary care providers, particularly dentists, should screen for sexual health issues as part of their routine oral health analysis. Mental Health and Sexual Health: Another featured study examines the relationship between mental health and risky sexual behaviors, providing evidence for why mental health professionals and oral health providers should be mindful of how mental health conditions can influence a person's sexual behavior and oral health outcomes. The figure includes a reference to HIV testing in the dental setting, advocating for oral health professionals to participate in public health efforts by offering HIV testing and education on safe sexual practices. The figure brings attention to how healthcare providers can use the information from these interrelated health domains to engage in better Outreach, Screening, and Management. The OSM Buddy system facilitates this process by promoting discussions between healthcare providers and patients that encompass oral, sexual, and mental health in an integrated manner. The figure underscores the importance of early identification of risk factors that may span across multiple areas of health, such as the oral manifestations of STIs (e.g., HPV-related oral cancers) or the impact of mental health disorders on sexual behavior and oral hygiene.

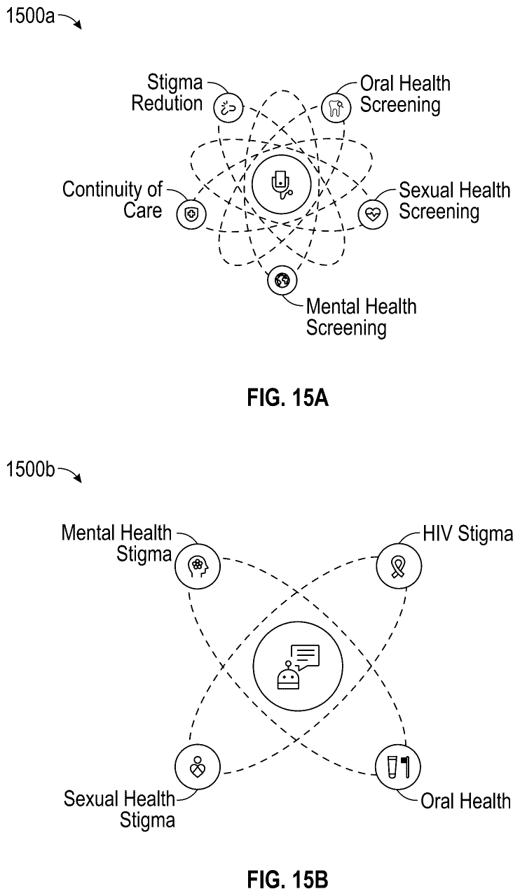

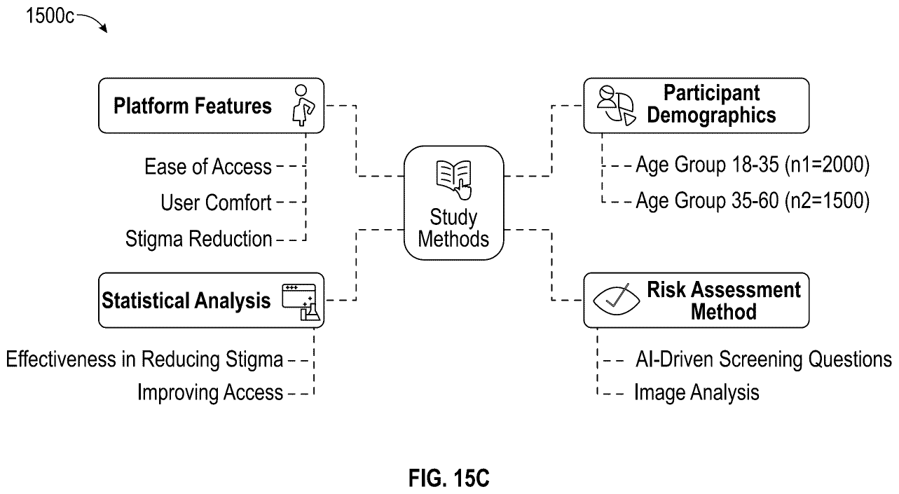

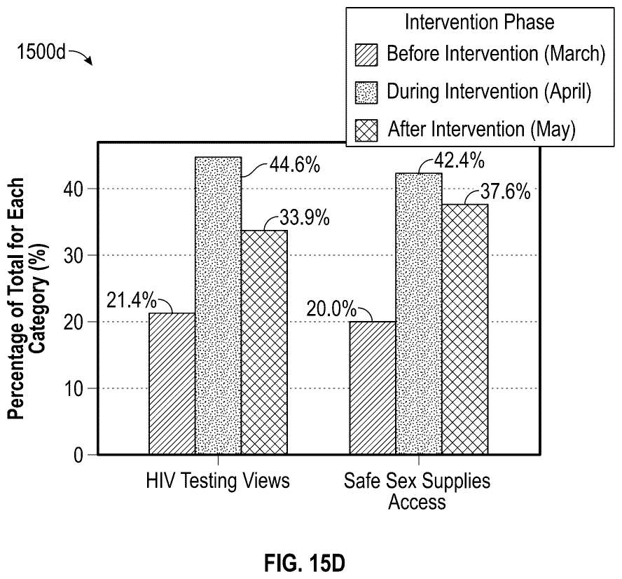

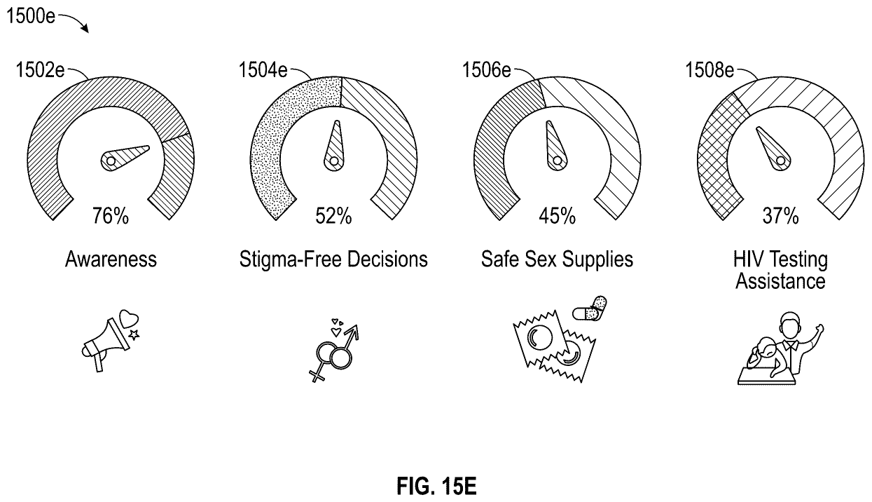

Referring to FIG. 15A illustrates an overview of the interconnected preventative health screenings and functionalities provided by the system, including oral health analysis, sexual health analysis, mental health analysis, stigma reduction, and continuity of care. The Diagram 1500a provides a visual representation of the healthcare analysis system's core functionalities and their interconnected nature. At the center, the diagram highlights the preventative health screening module as the central component through which users engage with various health-related analysis. Surrounding the central module are interconnected functionalities represented by orbiting pathways, which include: Oral Health analysis: Serves as a neutral, stigma-free entry point for broader health discussions and screenings. Sexual Health analysis: Facilitates risk analysis and healthcare recommendations related to sexual health conditions, such as HIV and STIs. Mental Health analysis: Enables users to assess behavioral and mental health conditions while identifying related risks. Stigma Reduction: Represents the system's gamification features, such as the Stigma Meter, which encourage user engagement by addressing and reducing stigmas. Continuity of Care: Highlights the integration of actionable healthcare recommendations, telehealth appointments, and care pathways that ensure ongoing and consistent healthcare management for the user. This figure visually conveys how the system integrates various health domains into a unified and interconnected analysis framework, improving accessibility and user experience.

Referring to FIG. 15B illustrates the stigma interplay across various health domains, including mental health stigma, HIV stigma, sexual health stigma, and oral health, and highlights how the system addresses these stigmas through user interactions. The Diagram 1500b illustrates the system's focus on addressing and reducing stigmas associated with health conditions. The central portion of the figure represents the intelligent virtual assistant (IVA) chatbot, which facilitates anonymous, judgment-free healthcare interactions. Four interconnected pathways extend from the central IVA chatbot, representing the primary stigma domains that the system aims to address: Mental Health Stigma: Emphasizes the system's ability to assess and address mental health challenges while promoting mental well-being. HIV Stigma: Focuses on reducing stigma related to HIV testing and prevention (e.g., PrEP/PEP) by providing confidential and supportive healthcare analysis. Sexual Health Stigma: Highlights the system's role in encouraging screenings and discussions around sexually transmitted infections (STIs) in a stigma-free environment. Oral Health: Represents the system's use of oral health as a stigma-free entry point, allowing users to engage with broader healthcare analysis discreetly. The pathways between these stigma domains reflect their interconnected nature, aligning with the system's syndemic evaluation framework. By addressing these stigmas collectively, the system promotes early intervention, informed decision-making, and improved health outcomes for the user.

Referring to FIG. 15C illustrates a flow diagram representing the study methods implemented for evaluating the system's platform features, participant demographics, risk analysis methods, and statistical analysis to assess the effectiveness of reducing stigma and improving healthcare access. The Diagram 1500c provides a flow diagram depicting the study methods employed to evaluate the effectiveness, user engagement, and accessibility of the healthcare analysis system. At the center of the diagram is the “Study Methods” component, which integrates four key elements: Platform Features, Participant Demographics, Risk analysis Methods, and Statistical Analysis. Each component is described below: Platform Features: This section highlights the features evaluated during the study to ensure the platform's effectiveness in user engagement and healthcare delivery. The platform features include: Ease of Access: The ability for users to easily navigate the system without requiring login credentials or personal data. User Comfort: The system's user-friendly design that prioritizes anonymity and discretion during sensitive health screenings. Stigma Reduction: The role of features such as the Stigma Meter and AI-driven tools in reducing stigma associated with health conditions, including HIV, mental health, and sexual health. Participant Demographics: This section outlines the demographic details of study participants, categorized into two age groups. Age Group 18-35: Representing younger adults (n=2000). Age Group 35-60: Representing middle-aged adults (n=1500). The study includes a diverse set of participants to evaluate the platform's inclusivity and effectiveness across different age groups. Risk analysis Method: This section describes the methodologies employed for assessing user health risks. The methods include: AI-Driven Screening Questions: Dynamic and personalized screening questions generated using Natural Language Processing (NLP) and Natural Language Understanding (NLU) to gather user inputs. Image Analysis: The process of analyzing uploaded images of oral and skin abnormalities using AI and computer vision algorithms to enhance health risk analysis. Statistical Analysis: This section focuses on the evaluation metrics and outcomes derived from the study.

Referring to FIG. 15D illustrates a bar chart representing the impact of an intervention phase on user participation across two categories, namely HIV testing views and access to safe sex supplies, measured over three distinct phases. The diagram 1500d presents a bar chart, labeled as 1500d, that demonstrates the effect of a targeted intervention on healthcare engagement by comparing percentages of user participation across three time phases: before intervention (March), during intervention (April), and after intervention (May). The vertical axis represents the percentage of total participation for each category, while the horizontal axis highlights two key metrics-HIV testing views and safe sex supplies access. For the category of HIV testing views, the percentage stood at 21.4% before the intervention in March, indicating a baseline level of engagement. During the intervention phase in April, the percentage increased significantly to 44.6%, reflecting the immediate impact of the intervention. After the intervention phase in May, the percentage slightly decreased to 33.9%, though it remained substantially higher than the initial baseline, signifying sustained user interest in HIV testing. Similarly, for the category of safe sex supplies access, the chart shows that participation was 20.0% before the intervention in March. During the intervention in April, access to supplies improved notably, rising to 42.4%, which highlights the success of the intervention in improving healthcare resource availability. Post-intervention, in May, the percentage slightly declined to 37.6%, but it still demonstrated a significant improvement over the pre-intervention levels. The bar chart distinguishes each phase with unique patterns: diagonal lines for March, cross-hatching for April, and dotted shading for May, providing a clear visual representation of the change over time. This figure effectively conveys how the intervention phase contributed to an increase in user participation and access to essential healthcare services, specifically HIV testing and safe sex supplies, and highlights the importance of such interventions in fostering positive behavioral changes and improved healthcare outcomes.

Referring to FIG. 15E illustrates a series of graphical gauges representing the outcomes of user engagement with the automated preventative health screening and data-driven health risk analysis system, showing percentages for key areas of improvement, including awareness, stigma-free decision-making, access to safe sex supplies, and HIV testing assistance. The diagram 1500e presents a graphical representation labeled as 1500e, showing four circular gauges that depict the percentages of progress achieved in critical health intervention areas facilitated by the automated preventative health screening and data-driven health risk analysis system. Each gauge corresponds to a specific outcome-awareness, stigma-free decisions, safe sex supplies, and HIV testing assistance-and includes an associated percentage that quantifies the user engagement or improvement in that area. The first gauge, labeled Awareness, displays a percentage of 76%, indicating that a significant majority of users experienced an increase in their overall health awareness as a result of system interventions. The second gauge, labeled Stigma-Free Decisions, shows a percentage of 52%, reflecting the system's effectiveness in encouraging users to make decisions related to healthcare, such as screenings and consultations, in a stigma-free environment. The third gauge, labeled Safe Sex Supplies, represents a 45% improvement in user access to critical healthcare resources like condoms and other safe sex supplies, highlighting the system's role in facilitating resource distribution. The fourth and final gauge, labeled HIV Testing Assistance, shows a 37% engagement rate, demonstrating the system's contribution to guiding users toward HIV testing services and encouraging them to seek assistance. Each gauge is visually represented with a segmented circular meter, where the shaded portion corresponds to the reported percentage. Additionally, icons placed below each gauge provide a visual cue for the corresponding outcome: a megaphone for awareness, a gender symbol for stigma-free decisions, condoms for safe sex supplies, and a healthcare provider assisting a user for HIV testing assistance. This figure highlights the measurable impact of the system across multiple healthcare outcomes, illustrating its ability to enhance awareness, promote stigma reduction, improve resource access, and facilitate HIV testing support.

In accordance with one or more exemplary embodiments of the present disclosure, the intelligent virtual assistant (IVA) chatbot dynamically adapts the screening questions based on real-time user inputs, whereby the chatbot enables intent-driven personalization to refine user engagement and ensures accurate health risk evaluations across diverse health domains, including oral health, behavioral health, sexual health, and mental health, thereby enhancing the precision and relevance of preventative health screening.

In accordance with one or more exemplary embodiments of the present disclosure, the server executes instructions from the healthcare data processing module, wherein the healthcare data processing module comprises a healthcare data analyzing module configured to analyze interconnected risks across oral health, sexual health, mental health, and behavioral health using a syndemic evaluation framework, whereby the syndemic evaluation framework evaluates the interplay between these health domains to identify complex risk patterns, thereby enabling comprehensive and accurate health risk analysis.

In accordance with one or more exemplary embodiments of the present disclosure, the processor executes instructions from the preventative health screening module, wherein the preventative health screening module comprises a user interface module configured to leverage oral health as a neutral and stigma-free entry point, whereby the user interface module facilitates the initiation of screenings for broader health risks, including sexual health, mental health, and substance use, thereby encouraging user participation and reducing stigma associated with health risk evaluations.

In accordance with one or more exemplary embodiments of the present disclosure, the processor executes instructions from the preventative health screening module, wherein the preventative health screening module comprises a question generating module configured to generate intent-driven and personalized screening questions using Natural Language Processing (NLP) and Natural Language Understanding (NLU) based on real-time user inputs, whereby the question generating module dynamically tailors the questions to user responses, thereby enhancing user engagement and ensuring accurate health risk analysis.

In accordance with one or more exemplary embodiments of the present disclosure, the server executes instructions from the healthcare data processing module, wherein the healthcare data processing module comprises an image processing and analysis module configured to analyze user-uploaded images using AI and computer vision algorithms, whereby the analysis enhances the precision of health risk assessments.

In accordance with one or more exemplary embodiments of the present disclosure, the server executes instructions from the healthcare data processing module, wherein the healthcare data processing module comprises a unique ID generation module configured to generate a secure alphanumeric identifier for each user upon completing the anonymous screening process, thereby enabling anonymous and secure engagement with the system.

In accordance with one or more exemplary embodiments of the present disclosure, the server executes instructions from the healthcare data processing module, wherein the healthcare data processing module comprises a language translation module configured to provide real-time translations and culturally sensitive phrasing for diverse user populations, thereby enhancing accessibility and user engagement.

In accordance with one or more exemplary embodiments of the present disclosure, the server executes instructions from the healthcare data processing module, wherein the healthcare data processing module comprises a report generating module configured to generate a PDF file containing details of the image risk score, oral risk score, mental risk score, sexual risk score, and overall risk score, thereby enabling comprehensive documentation of health assessments.

In accordance with one or more exemplary embodiments of the present disclosure, the report generating module is configured to generate actionable healthcare recommendations based on the processed user data, including eligibility checks for medications such as PrEP, PEP, and Naloxone, guided testing pathways, telehealth consultations, and access to peer support networks, thereby facilitating informed healthcare decisions.

In accordance with one or more exemplary embodiments of the present disclosure, the report generating module is configured to generate downloadable reports containing user risk scores, AI analysis results, and personalized recommendations for further healthcare actions, thereby improving user access to detailed health insights.

In accordance with one or more exemplary embodiments of the present disclosure, the server executes instructions from the healthcare data processing module, wherein the healthcare data processing module comprises an image processing and analysis module configured to perform image-based analysis of oral and skin abnormalities using AI and computer vision algorithms, thereby enhancing the precision of health risk scoring.

In accordance with one or more exemplary embodiments of the present disclosure, the processor executes instructions from the preventative health screening module, wherein the preventative health screening module comprises a delete module configured to enable the user to delete their information and close the chat at any time, thereby maintaining privacy and security.

In accordance with one or more exemplary embodiments of the present disclosure, the server executes instructions from the healthcare data processing module, wherein the healthcare data processing module comprises a healthcare data analyzing module configured to assess the interplay between communicable diseases, including HIV, STIs, and Mpox, and non-communicable conditions, including diabetes, hypertension, and mental health disorders, thereby facilitating comprehensive risk evaluations.

In accordance with one or more exemplary embodiments of the present disclosure, the processor executes instructions from the preventative health screening module, wherein the preventative health screening module comprises a user rewards module configured to track user actions and provide rewards for completing health-promoting activities, including screenings and consultations, thereby encouraging continued user engagement.

In accordance with one or more exemplary embodiments of the present disclosure, the processor executes instructions from the preventative health screening module, wherein the preventative health screening module comprises a machine-readable code access module configured to allow users to access the system by scanning QR codes distributed by participating organizations without requiring login credentials, thereby simplifying system accessibility.

In accordance with one or more exemplary embodiments of the present disclosure, the server executes instructions from the healthcare data processing module, wherein the healthcare data processing module comprises a geographic insights module configured to provide anonymized, location-based health insights derived from aggregated user screening data, thereby supporting population health analysis.

In accordance with one or more exemplary embodiments of the present disclosure, the server executes instructions from the healthcare data processing module, wherein the healthcare data processing module comprises a syndemic monitoring module configured to analyze aggregated and anonymized health data in real time to identify public health trends and syndemic patterns, thereby enabling proactive public health interventions.

In accordance with one or more exemplary embodiments of the present disclosure, the processor executes instructions from the preventative health screening module, wherein the preventative health screening module comprises a care assistance module configured to provide prescription reminders, eligibility form submissions, and guided workflows to streamline healthcare management, thereby improving user adherence to healthcare plans.

In accordance with one or more exemplary embodiments of the present disclosure, the server executes instructions from the healthcare data processing module, wherein the healthcare data processing module comprises an oral risk score generating module configured to generate the oral risk score of the user based on processed oral health information data, thereby enhancing oral health risk analysis.

In accordance with one or more exemplary embodiments of the present disclosure, the server executes instructions from the healthcare data processing module, wherein the healthcare data processing module comprises a mental risk score generating module configured to generate the mental risk score of the user based on processed behavioral health information data, thereby providing insights into mental health risks.

In accordance with one or more exemplary embodiments of the present disclosure, the server executes instructions from the healthcare data processing module, wherein the healthcare data processing module comprises a sexual risk score generating module configured to generate the sexual risk score of the user based on processed sexual life information, drug usage information, and sexual abuse information, thereby ensuring comprehensive sexual health risk evaluations.

In accordance with one or more exemplary embodiments of the present disclosure, the server executes instructions from the healthcare data processing module, wherein the healthcare data processing module comprises an overall risk score generating module configured to generate the overall risk score based on processed oral health information, behavioral health information, sexual life information, drug usage information, sexual abuse information, and image data of oral and skin abnormalities, thereby providing an integrated health risk assessment.

In accordance with one or more exemplary embodiments of the present disclosure, the second computing device executes instructions from the preventative health screening module, wherein the preventative health screening module comprises a care plan integration module configured to generate personalized care plans for healthcare providers, including case managers and providers, whereby the care plans enable timely and informed decisions, thereby improving the quality of care for users.

In accordance with one or more exemplary embodiments of the present disclosure, the second computing device executes instructions from the preventative health screening module, wherein the preventative health screening module comprises an authorized access module configured to provide a comprehensive view of a user's health data to case managers, healthcare providers, and other authorized professionals, thereby enabling informed decision-making based on comprehensive screening results.

In accordance with one or more exemplary embodiments of the present disclosure, the second computing device executes instructions from the preventative health screening module, wherein the preventative health screening module comprises the authorized access module configured to enable case managers and providers to verify their profiles through their organizations before accessing the preventative health screening module, thereby confirming that only authorized professionals use the module for case management and healthcare decisions.

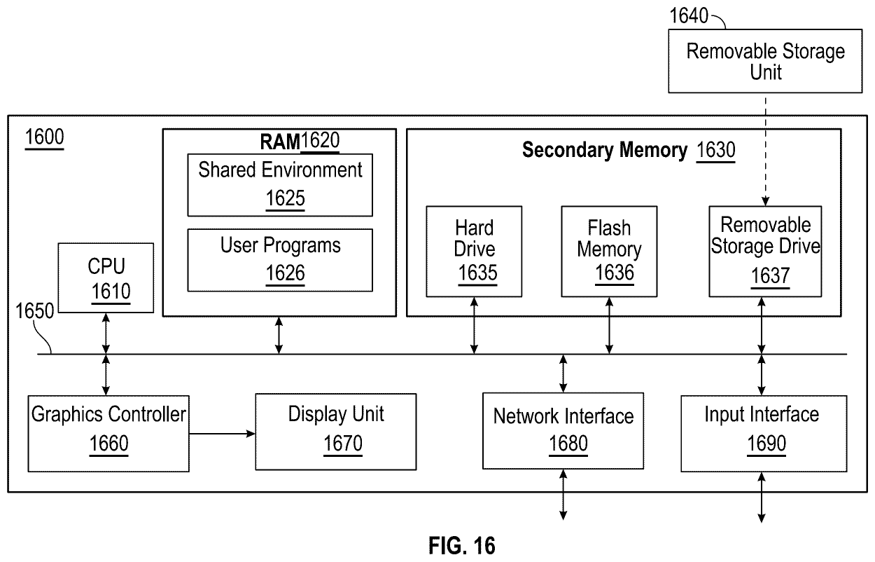

Referring to FIG. 16 is a block diagram 1600 illustrating the details of a digital processing system 1600 in which various aspects of the present disclosure are operative by execution of appropriate software instructions. The Digital processing system 1600 may correspond to the computing device (or any other system in which the various features disclosed above can be implemented).

Digital processing system 1600 may contain one or more processors such as a central processing unit (CPU) 1610, random access memory (RAM) 1620, secondary memory 1630, graphics controller 1660, display unit 1670, network interface 1680, and input interface 1690. All the components except display unit 1670 may communicate with each other over communication path 1650, which may contain several buses as is well known in the relevant arts. The components of FIG. 16 are described below in further detail.

CPU 1610 may execute instructions stored in RAM 1620 to provide several features of the present disclosure. CPU 1610 may contain multiple processing units, with each processing unit potentially being designed for a specific task. Alternatively, CPU 1610 may contain only a single general-purpose processing unit.

RAM 1620 may receive instructions from secondary memory 1530 using communication path 1650. RAM 1620 is shown currently containing software instructions, such as those used in threads and stacks, constituting shared environment 1625 and/or user programs 1626. Shared environment 1625 includes operating systems, device drivers, virtual machines, etc., which provide a (common) run time environment for execution of user programs 1626.

Graphics controller 1660 generates display signals (e.g., in RGB format) to display unit 1670 based on data/instructions received from CPU 1610. Display unit 1570 contains a display screen to display the images defined by the display signals. Input interface 1690 may correspond to a keyboard and a pointing device (e.g., touchpad, mouse) and may be used to provide inputs. Network interface 1680 provides connectivity to a network (e.g., using Internet Protocol), and may be used to communicate with other systems (such as those shown in FIG. 1) connected to the network.

Secondary memory 1630 may contain hard drive 1635, flash memory 1536, and removable storage drive 1637. Secondary memory 1630 may store the data software instructions (e.g., for performing the actions noted above with respect to the Figures), which enable digital processing system 1600 to provide several features in accordance with the present disclosure.

Some or all of the data and instructions may be provided on removable storage unit 1640, and the data and instructions may be read and provided by removable storage drive 1637 to CPU 1610. Floppy drive, magnetic tape drive, CD-ROM drive, DVD Drive, Flash memory, removable memory chip (PCMCIA Card, EEPROM) are examples of such removable storage drive 1637.

Removable storage unit 1640 may be implemented using medium and storage format compatible with removable storage drive 1637 such that removable storage drive 1637 can read the data and instructions. Thus, removable storage unit 1640 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 1640 or hard disk installed in hard drive 1635. These computer program products are means for providing software to digital processing system 1500. CPU 1610 may retrieve the software instructions and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 16530. Volatile media includes dynamic memory, such as RAM 1520. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus (communication path) 1650. Transmission media can also take the form of acoustic or light waves, such as those generated during radio-wave and infra-red data communications.

Reference throughout this specification to “one embodiment”, “an embodiment”, or similar language means that a particular feature, structure, or characteristic described in connection with the embodiment is included in at least one embodiment of the present disclosure. Thus, appearances of the phrases “in one embodiment”, “in an embodiment” and similar language throughout this specification may, but do not necessarily, all refer to the same embodiment.

Furthermore, the described features, structures, or characteristics of the disclosure may be combined in any suitable manner in one or more embodiments. In the above description, numerous specific details are provided such as examples of programming, software modules, user selections, network transactions, database queries, database structures, hardware modules, hardware circuits, hardware chips, etc., to provide a thorough understanding of embodiments of the disclosure.

Although the present disclosure has been described in terms of certain preferred embodiments and illustrations thereof, other embodiments and modifications to preferred embodiments may be possible that are within the principles of the invention. The above descriptions and figures are therefore to be regarded as illustrative and not restrictive.

Thus the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. An artificial intelligence-based system for automated preventative health screening and data-driven health risk analysis, comprising:
a first computing device comprising a processor configured to execute instructions from a preventative health screening module, wherein the preventative health screening module includes an intelligent virtual assistant (IVA) chatbot configured to interact with a user through a dynamic and AI-driven interface, whereby the intelligent virtual assistant (IVA) chatbot generates a series of intent-driven and personalized screening questions using Natural Language Processing (NLP) and Natural Language Understanding (NLU), thereby facilitating user engagement;
wherein the intelligent virtual assistant (IVA) chatbot enables the user to respond to the screening questions by selecting predefined options, providing textual responses, and uploading images of oral and skin abnormalities for AI-based analysis, whereby the preventative health screening module collects the user responses and image data related to oral and skin abnormalities and transmits the collected data to a server over a network;
the server comprising a healthcare data processing module configured to receive and process the user responses and image data using AI-driven algorithms and a syndemic evaluation framework, wherein the syndemic evaluation framework evaluates the interplay between communicable diseases, including HIV, STIs, and Mpox, and non-communicable conditions, including diabetes, hypertension, and mental health disorders, whereby the healthcare data processing module generates personalized healthcare recommendations and numerical risk scores related to oral health, sexual health, mental health, and overall health using a proprietary scoring mechanism, and transmits the generated recommendations and risk scores to the first computing device;
wherein the preventative health screening module on the first computing device displays the numerical risk scores and personalized healthcare recommendations to the user, and presents additional actionable options, including booking telehealth appointments, ordering home test kits, connecting with healthcare providers, and accessing rewards for health-promoting actions, whereby the preventative health screening module provides a gamification feature with a stigma meter, wherein the stigma meter visually represents the user's progress in overcoming stigma, dynamically updates based on user actions such as completing screenings, ordering test kits, and engaging with support resources, and provides rewards, including discounts, coupons, and gamified perks, thereby encouraging continued user engagement;
a second computing device and a third computing device communicatively connected to the server over the network, wherein the second computing device and the third computing device comprise the preventative health screening module configured to enable healthcare providers and case managers to securely access user-approved health data, including numerical risk scores, image analysis results, and personalized healthcare recommendations, whereby the second and third computing devices allow healthcare providers and case managers to make decisions and develop personalized care plans while maintaining user anonymity.

2. The system of claim 1, wherein the intelligent virtual assistant (IVA) chatbot dynamically adapts the screening questions based on real-time user inputs, whereby the chatbot enables intent-driven personalization to refine user engagement and ensures accurate health risk evaluations across diverse health domains, including oral health, behavioral health, sexual health, and mental health, thereby enhancing the precision and relevance of preventative health screening.

3. The system of claim 1, wherein the server executes instructions from the healthcare data processing module, wherein the healthcare data processing module comprises a healthcare data analyzing module configured to analyze interconnected risks across oral health, sexual health, mental health, and behavioral health using a syndemic evaluation framework, whereby the syndemic evaluation framework evaluates the interplay between these health domains to identify complex risk patterns, thereby enabling comprehensive and accurate health risk analysis.

4. The system of claim 1, wherein the processor executes instructions from the preventative health screening module, wherein the preventative health screening module comprises a user interface module configured to leverage oral health as a neutral and stigma-free entry point, whereby the user interface module facilitates the initiation of screenings for broader health risks, including sexual health, mental health, and substance use, thereby encouraging user participation and reducing stigma associated with health risk evaluations.

5. The system of claim 1, wherein the processor executes instructions from the preventative health screening module, wherein the preventative health screening module comprises a question generating module configured to generate intent-driven and personalized screening questions using Natural Language Processing (NLP) and Natural Language Understanding (NLU) based on real-time user inputs, whereby the question generating module dynamically tailors the questions to user responses, thereby enhancing user engagement and ensuring accurate health risk assessment.

6. The system of claim 1, wherein the server executes instructions from the healthcare data processing module, wherein the healthcare data processing module comprises an image processing and analysis module configured to analyze user-uploaded images using AI and computer vision algorithms, whereby the analysis enhances the precision of health risk assessments.

7. The system of claim 1, wherein the server executes instructions from the healthcare data processing module, wherein the healthcare data processing module comprises a unique ID generation module configured to generate a secure alphanumeric identifier for each user upon completing the anonymous screening process, thereby enabling anonymous and secure engagement with the system.

8. The system of claim 1, wherein the server executes instructions from the healthcare data processing module, wherein the healthcare data processing module comprises a language translation module configured to provide real-time translations and culturally sensitive phrasing for diverse user populations, thereby enhancing accessibility and user engagement.

9. The system of claim 1, wherein the server executes instructions from the healthcare data processing module, wherein the healthcare data processing module comprises a report generating module configured to generate a PDF file containing details of the image risk score, oral risk score, mental risk score, sexual risk score, and overall risk score, thereby enabling comprehensive documentation of health assessments.

10. The system of claim 9, wherein the report generating module is configured to generate actionable healthcare recommendations based on the processed user data, including eligibility checks for medications such as PrEP, PEP, and Naloxone, guided testing pathways, telehealth consultations, and access to peer support networks, thereby facilitating informed healthcare decisions.

11. The system of claim 9, wherein the report generating module is configured to generate downloadable reports containing user risk scores, AI analysis results, and personalized recommendations for further healthcare actions, thereby improving user access to detailed health insights.

12. The system of claim 1, wherein the server executes instructions from the healthcare data processing module, wherein the healthcare data processing module comprises an image processing and analysis module configured to perform image-based analysis of oral and skin abnormalities using AI and computer vision algorithms, thereby enhancing the precision of health risk scoring.

13. The system of claim 1, wherein the processor executes instructions from the preventative health screening module, wherein the preventative health screening module comprises a delete module configured to enable the user to delete their information and close the chat at any time, thereby maintaining privacy and security.

14. The system of claim 1, wherein the server executes instructions from the healthcare data processing module, wherein the healthcare data processing module comprises a healthcare data analyzing module configured to assess the interplay between communicable diseases, including HIV, STIs, and Mpox, and non-communicable conditions, including diabetes, hypertension, and mental health disorders, thereby facilitating comprehensive risk evaluations.

15. The system of claim 1, wherein the processor executes instructions from the preventative health screening module, wherein the preventative health screening module comprises a user rewards module configured to track user actions and provide rewards for completing health-promoting activities, including screenings and consultations, thereby encouraging continued user engagement.

16. The system of claim 1, wherein the processor executes instructions from the preventative health screening module, wherein the preventative health screening module comprises a machine-readable code access module configured to allow users to access the system by scanning QR codes distributed by participating organizations without requiring login credentials, thereby simplifying system accessibility.

17. The system of claim 1, wherein the server executes instructions from the healthcare data processing module, wherein the healthcare data processing module comprises a geographic insights module configured to provide anonymized, location-based health insights derived from aggregated user screening data, thereby supporting population health analysis.

18. The system of claim 1, wherein the server executes instructions from the healthcare data processing module, wherein the healthcare data processing module comprises a syndemic monitoring module configured to analyze aggregated and anonymized health data in real time to identify public health trends and syndemic patterns, thereby enabling proactive public health interventions.

19. The system of claim 1, wherein the processor executes instructions from the preventative health screening module, wherein the preventative health screening module comprises a care assistance module configured to provide prescription reminders, eligibility form submissions, and guided workflows to streamline healthcare management, thereby improving user adherence to healthcare plans.

20. The system of claim 1, wherein the server executes instructions from the healthcare data processing module, wherein the healthcare data processing module comprises an oral risk score generating module configured to generate the oral risk score of the user based on processed oral health information data, thereby enhancing oral health risk analysis.

21. The system of claim 1, wherein the server executes instructions from the healthcare data processing module, wherein the healthcare data processing module comprises a mental risk score generating module configured to generate the mental risk score of the user based on processed behavioral health information data, thereby providing insights into mental health risks.

22. The system of claim 1, wherein the server executes instructions from the healthcare data processing module, wherein the healthcare data processing module comprises a sexual risk score generating module configured to generate the sexual risk score of the user based on processed sexual life information, drug usage information, and sexual abuse information, thereby ensuring comprehensive sexual health risk evaluations.

23. The system of claim 1, wherein the server executes instructions from the healthcare data processing module, wherein the healthcare data processing module comprises an overall risk score generating module configured to generate the overall risk score based on processed oral health information, behavioral health information, sexual life information, drug usage information, sexual abuse information, and image data of oral and skin abnormalities, thereby providing an integrated health risk assessment.

24. The system of claim 1, wherein the second computing device executes instructions from the preventative health screening module, wherein the preventative health screening module comprises a care plan integration module configured to generate personalized care plans for healthcare providers, including case managers and providers, whereby the care plans enable timely and informed decisions, thereby improving the quality of care for users.

25. The system of claim 1, wherein the second computing device executes instructions from the preventative health screening module, wherein the preventative health screening module comprises an authorized access module configured to provide a comprehensive view of a user's health data to case managers, healthcare providers, and other authorized professionals, thereby enabling informed decision-making based on comprehensive screening results.

26. The system of claim 1, wherein the second computing device executes instructions from the preventative health screening module, wherein the preventative health screening module comprises the authorized access module configured to enable case managers and providers to verify their profiles through their organizations before accessing the preventative health screening module, thereby confirming that only authorized professionals use the module for case management and healthcare decisions.

27. A method for automated preventative health screening and data-driven health risk analysis, comprising:
enabling a user to initiate an anonymous screening process through a preventative health screening module enabled on a first computing device, wherein the preventative health screening module presents an intelligent virtual assistant (IVA) chatbot configured to allow the user to conduct the anonymous screening process without providing personal information, whereby the IVA chatbot generates intent-driven and personalized screening questions related to oral health, behavioral health, sexual health, mental health, substance use, and sexual abuse using Natural Language Processing (NLP) and Natural Language Understanding (NLU);
allowing the user to respond to the screening questions through the IVA chatbot by selecting predefined options, providing textual responses, and uploading images of oral and skin abnormalities for AI-based analysis, whereby the preventative health screening module collects the user's responses and image data and transmits the collected data to a server over a network;
receiving the user responses and image data by a healthcare data processing module enabled on the server, wherein the healthcare data processing module processes the received data using AI-driven algorithms and a syndemic evaluation framework to evaluate the interplay between communicable diseases and non-communicable conditions, thereby enabling comprehensive health risk analysis;
generating personalized healthcare recommendations and numerical risk scores for oral, sexual, mental, and overall health by the healthcare data processing module using a proprietary scoring mechanism, and transmitting the personalized healthcare recommendations and numerical risk scores to the first computing device over the network;
displaying the numerical risk scores, personalized healthcare recommendations, and additional actionable options for the user on the first computing device by the preventative health screening module, wherein the actionable options include booking telehealth appointments, ordering home test kits, connecting with healthcare providers, and accessing rewards for health-promoting activities, whereby the preventative health screening module dynamically updates the user's progress using a stigma meter that visually represents the user's progress in overcoming stigma; and
enabling healthcare providers and case managers to securely access user-approved health data through a second computing device and a third computing device over the network, wherein the healthcare data includes numerical risk scores, AI-based image analysis results, and personalized healthcare recommendations, whereby the second and third computing devices allow healthcare providers and case managers to make informed decisions and develop personalized care plans while maintaining user anonymity, thereby improving the quality of care and ensuring secure engagement with the system.

28. A computer program product comprising a non-transitory computer-readable medium having a computer-readable program code embodied therein to be executed by one or more processors, said program code including instructions to:
enable a user to initiate an anonymous screening process through a preventative health screening module enabled on a first computing device, wherein the preventative health screening module presents an intelligent virtual assistant (IVA) chatbot configured to allow the user to conduct the anonymous screening process without providing personal information, whereby the IVA chatbot generates intent-driven and personalized screening questions related to oral health, behavioral health, sexual health, mental health, substance use, and sexual abuse using Natural Language Processing (NLP) and Natural Language Understanding (NLU);
allow the user to respond to the screening questions through the IVA chatbot by selecting predefined options, providing textual responses, and uploading images of oral and skin abnormalities for AI-based analysis, whereby the preventative health screening module collects the user's responses and image data and transmits the collected data to a server over a network;
receive the user responses and image data by a healthcare data processing module enabled on the server, wherein the healthcare data processing module processes the received data using AI-driven algorithms and a syndemic evaluation framework to evaluate the interplay between communicable diseases and non-communicable conditions, thereby enabling comprehensive health risk analysis;
generate personalized healthcare recommendations and numerical risk scores for oral, sexual, mental, and overall health by the healthcare data processing module using a proprietary scoring mechanism, and transmitting the personalized healthcare recommendations and numerical risk scores to the first computing device over the network;
display the numerical risk scores, personalized healthcare recommendations, and additional actionable options for the user on the first computing device by the preventative health screening module, wherein the actionable options include booking telehealth appointments, ordering home test kits, connecting with healthcare providers, and accessing rewards for health-promoting activities, whereby the preventative health screening module dynamically updates the user's progress using a stigma meter that visually represents the user's progress in overcoming stigma; and
enable healthcare providers and case managers to securely access user-approved health data through a second computing device and a third computing device over the network, wherein the healthcare data includes numerical risk scores, AI-based image analysis results, and personalized healthcare recommendations, whereby the second and third computing devices allow healthcare providers and case managers to make informed decisions and develop personalized care plans while maintaining user anonymity, thereby improving the quality of care and ensuring secure engagement with the system.
